(19) United States
(12) Patent Application Publication
(10) Pub. No.: US 2005/0085498 A1
(43) Pub. Date: Apr. 21, 2005

(54) Oral formulation of lipid soluble thiamine, lipoic acid, creatine derivative, and L-arginine alpha-ketoglutarate

(76) Inventor: Edward A. Byrd, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/947,054

(22) Filed: Sep. 21, 2004

Related U.S. Application Data
(63) Continuation-in-part of application No. 10/693,837, filed on Oct. 23, 2003.
Application No. 10/693,837 is a continuation-in-part of application No. 10/412,559, filed on Apr. 11, 2003.
Application No. 10/412,559 is a continuation of application No. 09/755,890, filed on Jan. 5, 2001, now Pat. No. 6,572,888.
Application No. 09/755,890 is a continuation-in-part of application No. 09/288,245, filed on Apr. 8, 1999, now Pat. No. 6,197,340.
Application No. 09/755,890 is a continuation-in-part of application No. 09/112,623, filed on Jul. 9, 1998, now abandoned.
(60) Provisional application No. 60/102,605, filed on Oct. 1, 1998.
(60) Provisional application No. 60/087,203, filed on May 28, 1998.

(51) Int. Cl. A61K 31/555; A61K 31/385; A61K 31/198

(52) U.S. Cl. 514/275; 514/440; 514/563; 514/565

(57) ABSTRACT

A formulation comprised of four active components which are a lipid soluble thiamine, lipoic acid, arginine α-ketoglutarate, and a creatine derivative for oral administration is disclosed. The active components may be combined with excipient materials in such a way that those materials provide for an immediate release of a first portion of the active ingredients from the formulation following by a gradual release of any remaining active ingredients in a manner which makes it possible to (1) quickly obtain a therapeutic level of the active ingredients; and (2) substantially increase the period of time over which therapeutic levels of the active ingredients are maintained relative to a quick release formulation. These features make it possible to use the formulation to obtain a range of beneficial effects including reducing serum glucose levels and maintaining those reduced glucose levels over time to treat diabetic polyneuropathy as well as improving circulation and increasing muscle performance.

### CROSS-REFERENCE TO RELATED APPLICATIONS

This application is a continuation-in-part of earlier filed U.S. patent application Ser. No. 10/693,837, filed Oct. 23, 2003 which is a continuation-in-part application of earlier filed U.S. patent application Ser. No. 10/412,559, filed Apr. 11, 2003 which is a continuation of Ser. No. 09/755,890, filed Jan. 5, 2001 (now issued U.S. Pat. No. 6,572,888 issued Jun. 3, 2003) which is a continuation-in-part of earlier filed patent application Ser. No. 09/288,245, filed Apr. 8, 1999 (now issued U.S. Pat. No. 6,197,340 issued Mar. 6, 2001), which claims benefit of earlier filed provisional patent application Ser. No. 60/102,605, filed Oct. 1, 1998 and is a continuation-in-part of earlier filed patent application Ser. No. 09/112,623, filed Jul. 9, 1998, which is the converted patent application of provisional patent application Ser. No. 60/087,203, filed May 28, 1998 to which we claim priority under 35 U.S:C. §120 and §119(e) each of which is incorporated herein by reference in their entirety.

### FIELD OF THE INVENTION

The invention relates generally to oral formulations which may be a controlled release oral formulation of pharmaceutically active compounds and methods of treatment using the formulations. More particularly, the invention relates to an oral formulation of a lipid soluble thiamine, lipoic acid, a creatine derivative and L-arginine α-ketoglutarate.

### BACKGROUND OF THE INVENTION

### Thiamine

Vitamin B1 (Thiamine or thiamin), the first B vitamin benefits the nervous system and mental attitude. Its odor and flavor are similar to those of yeast. Thiamine can be destroyed by the cooking process, especially by boiling or moist heat, but less by dry heat, such as baking.

Like most other B vitamins, thiamine is needed in regular supply, though after its absorption from the upper and lower small intestine, some B1 is stored in the liver, heart, and kidneys. Most excess thiamine is eliminated in the urine; some seems to be excreted in the sweat as well.

Since thiamine is lost in cooking and is depleted by use of sugar, coffee, tannin from black teas, nicotine, and alcohol, it is necessary to insure that intake of thiamine is optimal. There are a number of food sources for thiamine; however, they may not be the everyday fare for many people. Good sources of vitamin B1 include the germ and bran of wheat, rice husks (outer covering), and the outer portion of other grains. With the milling of grains and use of refined flours and white or “polished” rice, many of us are no longer getting the nourishment of thiamine that is available when we eat wholesome, unprocessed foods.

Other good sources of thiamine besides wheat germ and bran, whole wheat or enriched wheat flour, and brown rice are brewer's yeast and blackstrap molasses. Oats and millet have modest amounts, as do many vegetables, such as spinach and cauliflower, most nuts, sunflower seeds, and legumes, such as peanuts, peas, and beans. Of the fruits, avocado is the highest in vitamin B1. Pork has a high amount of this B vitamin. Many dried fruits contain some thiamine, though the sulfur dioxide often added as a preservative seems to destroy this vitamin.

Thiamine helps a great many bodily functions, acting as the coenzyme thiamine pyrophosphate (TPP). It has a key metabolic role in the cellular production of energy, mainly in glucose metabolism. Thiamine is also needed to metabolize ethanol, converting it to carbon dioxide and water. B1 helps in the initial steps of fatty acid and sterol production. In this way, thiamine also helps convert carbohydrate to fat for storage of potential energy.

Thiamine is important to the health of the nerves and nervous system, possibly because of its role in the synthesis of acetylcholine (via the production of acetyl CoA), an important neurotransmitter. With a lack of vitamin B1, the nerves are more sensitive to inflammation. Thiamine is linked to individual learning capacity and to growth in children. It is also important to the muscle tone of the stomach, intestines, and heart because of the function of acetylcholine at nerve synaptic junction. It is conceivable that adequate thiamine levels may help prevent the accumulation of fatty deposits in the arteries and thereby reduce the progression of atherosclerosis.

Thiamine is used to treat any of the symptoms of its deficiency or its deficiency disease beriberi (discussed below). It is used in the treatment of fatigue, irritability, low morale, and depression and to prevent air- or seasickness. It is beneficial to the nerves, heart, and muscular system function well. By aiding hydrochloric acid production, thiamine may help digestion or reduce nausea, and it can remedy constipation by increasing intestinal muscle tone. Thiamine is used commonly to improve healing after dental (or, often, any) surgery.

Increased thiamine intake may be administered for numerous mental illnesses and problems that affect the nerves. These include alcoholism and its nerve problems, multiple sclerosis, Bell's palsy (a facial nerve paralysis), and neuritis. Treatment with thiamine, for example, has been helpful in decreasing the sensory neuropathy that accompanies diabetes and in lessening the pain of trigeminal neuralgia. Thiamine also has a mild diuretic effect and is supportive of heart function, so it is suggested in the treatment program for many cardiovascular problems.

Lipid soluble forms of thiamine include benfotiamine and prosultiamine. When these compounds are orally administered they provide greater bioavailability as compared to water soluble versions of conventional thiamine (see Greg et al., Internation. J. Clinical Pharm. And Therapeutics, Vol. 36, No. 4, pages 216-221 (1998)) Benfotiamine in combination with vitamine B has been used in the treatment of diabetic polyneuropathy. (See Stracke et al., Exp. Clin. Endocrinal Diabetes, vol. 104, pages 311-316 (1996)).

### Lipoic Acid

A compound known as α-lipoic acid was first isolated by Reed and coworkers as an acetate replacing factor. It is slightly soluble in water, and soluble in organic solvents. α-lipoic acid is a chiral molecule and is known by a variety of names, including thioctic acid; 1,2-diethylene-3 pentanoic acid; 1,2-diethylene-3 valeric acid; and 6,8-thioctic acid. α-lipoic acid was tentatively classified as a vitamin after its isolation, but it was later found to be synthesized by animals and humans. The complete enzyme pathway that is responsible for the de novo synthesis has not yet been definitively elucidated. Several studies indicate that octanoate serves as the immediate precursor for the 8-carbon fatty acid chain, and cysteine appears to be the source of sulfur. As a lipoamide, it functions as a cofactor in the multienzyme complexes that catalyze the oxidative decarboxylation of α-keto acids such as pyruvate, α-keto glutarate, and branched chain α-keto acids.

More recently, a great deal of attention has been given to possible antioxidant functions for α-lipoic acid, and its reduced form, dihydrolipoic acid (DHLA). Lipoate, or its reduced form, DHLA, reacts with reactive oxygen species such as superoxide radicals, hydroxyl radicals, hypochlorous acid, peroxyl radicals, and singlet oxygen. It also protects membranes by interacting with vitamin C and glutathione, which may in turn recycle vitamin E. In addition to its antioxidant activities, DHLA may exert prooxidant actions to reduction of iron. α-lipoic acid administration has been shown to be beneficial in a number of oxidative stress models such as ischemia-reperfussion injury (IRI), diabetes (both α-lipoic acid and DHLA exhibit hydrophobic binding to proteins such as albumin, which can prevent glycation reactions), cataract formation, HIV activation, neurodegeneration, and radiation injury. Furthermore, lipoate can function as a redox regulator of proteins such as myoglobin, prolactin, thioredoxin, and NF-κB transcription factor.

Lipoate may also have other activities. For example, DHLA has been found in vitro to be an anti-inflammatory agent which at the same time interferes with nitric oxide release from inflammatory macrophages and protects target cells from oxygen radical attack. V. Burkhart, Dihydrolipoic Acid Protects Pancreatic Islet Cells from Inflammatory Attack, Agents Actions 38:60 (1993). This document, and all other documents cited to herein, is incorporated by reference as if reproduced fully herein.

Lipoic acid is a coenzyme for several enzymes. Lipoic acid is a coenzyme for both α-keto acid dehydrogenase complex enzymes (i.e. pyruvate dehydrogenase complex and α-keto glutarate dehydrogenase complex), branched chain α-keto acid dehydrogenase complex, and the glycine cleavage system. In the enzyme system, the body forms a multi-enzyme complex involving lipoic acid, that breaks down molecules of pyruvate produced in earlier metabolism, to form slightly smaller, high energy molecules, called acetyl-coenzyme A. This results in molecules that can enter into a series of reactions called the citric acid cycle, or Krebs cycle, which finishes the conversion of food into energy. Essentially, lipoic acid stimulates basal glucose transport and has a positive effect on insulin stimulated glucose uptake.

### Creatine

Creatine is an endogenous nutrient produced naturally by the liver in most vertebrates. The uses of creatine are many, including use as a supplement to increase muscle mass and enhance muscle performance as well as in emerging applications in the treatment of neuromuscular disorders.

Creatine, or N-(aminoiminomethyl)-N-methylglycine, is a sarcosine derivative present in the muscle tissue of many vertebrates, including man. Creatine is a central component of the metabolic system, and is involved in the provision of energy for work and exercise performance. Phosphocreatine (also known as creatine phosphate and phosphoryl creatine) helps to regenerate Adenosine TriPhosphate (ATP) during short bursts of high intensity exercise, and it has been found that the depletion of phosphocreatine has been associated with the onset of fatigue. It has also been discovered that the phosphocreatine pool in skeletal muscle is expandable. This has led to the oral supplementation of creatine and phosphocreatine to increase the levels; of these components in muscle, to thereby enhance exercise performance during intermittent activities that require strength and power. WO 94/02127, published on Feb. 3, 1994, discloses the use of creatine, optionally combined with amino acids or other components, in order to increase the muscle performance in mammals.

Creatine is synthesized from amino acids in the liver, pancreas and kidney, by the transfer of the guanidine moiety of arginine to glycine, which is then methylated to form creatine. Creatine which is synthesized in the liver, pancreas and kidney, is released into the bloodstream and actively taken up by the muscle cells, using the Na+ gradient. Creatine oral supplementation has been used to increase creatine and creatine phosphate stores, which are needed for high energy phosphorus metabolism. Recovery after high intensity exercise involves a resynthesis of phosphocreatine, which occurs via an oxygen-dependent process with half-life of about 30 seconds. During short-term high intensity intermittent exercise, the active muscles rely heavily on phosphocreatine for production of ATP. The rate of phosphocreatine resynthesis can be accelerated by the use of creatine supplementation in subjects who demonstrated an increase in creatine concentration. The benefits of creatine supplementation are particularly evident in high intensity activities that are intermittent in nature.

The creatine transport protein has an increased affinity for creatine and concentrates creatine within the cell. Once inside the cell, very little creatine is lost (approximately 2 grams per day in a 70 kg male). Based upon this information, it follows that small increases of plasma creatine (which can occur with creatine supplementation) result in increased transport activity. The loss of creatine from skeletal muscle is typically about 3% per day, which closely matches the amount of creatinine non-enzymatically produced by living human muscle. The main mechanism by which creatine is lost, is the conversion of creatine to creatinine, which is an irreversible non-enzymatic process. Thus, creatine lost from a cell is considered to be negligible, and the concentration of creatine in the cell is not at risk of depletion by virtue of exercise. Thus, the main advantage of creatine administration is in the fact that cellular creatine concentration is stable and not prone to being lost.

The most commonly used creatine supplement for oral consumption, is creatine monohydrate. Body builders find that shortly after beginning the use of creatine as a nutritional supplement, muscles take on additional mass and definition. Thus creatine supplements are becoming more popular as a steroid-free means of improving athletic performance and strength. Increasing the creatine in a diet through supplementation may therefore be useful to increase the blood plasma level of creatine and thus increase the amount of creatine in the muscles.

Creatine monohydrate is most commonly sold as a nutritional supplement in powder form. The powder may be blended with juices or other fluids, and then ingested. Prompt ingestion is important, because creatine is not stable in acidic solutions, such as juices. If creatine is retained in acidic solutions for even relatively short periods of time, most or all of the creatine in this solution converts to creatinine, which does not have the beneficial effects of creatine.

Creatine monohydrate supplementation at a dosage of 20 grams per day for a 5 day period has been the standard used during most studies in humans. Conventionally, creatine monohydrate is dissolved in approximately 300 milliliters of warm to hot water, the increased water temperature thereby increasing the solubility of creatine monohydrate. It has been found that creatine is not decomposed in the alimentary tract after oral administration, since there is no appreciable increase in urinary urea or ammonia. The results obtained for the conversion of retained creatine to creatinine have led researchers to believe that creatine is completely absorbed from the alimentary tract, then carried to the tissues, and hence either stored in the tissues or immediately rejected and eliminated by way of the kidneys.

Another problem with existing creatine supplementation is in the ability to provide consistent uniform results. It is believed that these inconsistent results arise because of the current methods of delivering creatine to the human body area. Current creatine oral supplementation, as discussed above relies on the use of creatine in powder form, which is dissolved in water and then taken orally. However, creatine in powder form does not dissolve well in water or other neutral pH liquids. The solubility of creatine in water is low, about 1 g in 75 ml. To obtain 10 grams, a subject would have to consume almost a liter of liquid. While increasing the temperature of the water increases the solubility of creatine monohydrate, there still is no consistency in the amount of creatine that is effectively dissolved in the water. For this reason, the consumer will take in varying amounts of creatine when consuming creatine monohydrate powder dissolved in water or other liquids.

Typically, creatine is taken up into muscle cells by specific transport proteins, the creatine transporter, and converted to phosphocreatine by creatine kinase. Muscle cells, including skeletal muscle and the heart muscle, function by utilizing cellular energy released from the conversion of adenosine triphosphate (ATP) to adenosine diphosphate (ADP). The amount of phosphocreatine in the muscle cell determines the amount of time it will take for the muscle to recover from activity and regenerate adenosine triphosphate (ATP). Phosphocreatine is a rapidly accessible source of phosphate required for regeneration of adenosine triphosphate (ATP) and sustained use of the muscle.

For example, energy used to expand and contract muscles is supplied from adenosine triphosphate (ATP). Adenosine triphosphate (ATP) is metabolized in the muscle by cleaving a phosphate radical to release energy needed to contract the muscle. Adenosine diphosphate (ADP) is formed as a byproduct of this metabolism. The most common sources of adenosine triphosphate (ATP) are from glycogen and creatine phosphate. Creatine phosphate is favored as a ready source of phosphate because it is able to resynthesize adenosine triphosphate (ATP) at a greater rate than is typically achieved utilizing glycogen. Therefore, increasing the amount of creatine in the muscle increases the muscle stores of phosphocreatine and has been proven to increase muscle performance and increase muscle mass.

However, creatine itself is poorly soluble in an aqueous solution. Further, creatine is not well absorbed from the gastrointestinal (GI) tract, which has been estimated to have a 1 to 14 percent absorption rate. Thus, current products require large amounts of creatine to be administered to be effective, typically 5 grams or more. Additionally, side effects such as bloating, gastrointestinal (GI) distress, diarrhea, and the like are encountered with these high dosages.

Therefore, it would be desirable to provide an improved approach for enhancing absorption of creatine.

### Arginine α-Ketoglutarate

Arginine α-ketoglutarate, also known as arginine 2-oxoglutarate, is an organic salt which possesses a number of physiological uses. Studies conducted in 1977 revealed its ability to enhance hepatic detoxification capacity when administered in high dosage to patients with liver cirrhosis. (Muting et al. (1977) MMW Munch Med Wochenschr, 119(16):535-8.) Its effects were marked by a significant decrease in the level of plasma ammonia and free serum phenols, which indicate improved oxidative decomposition of these compounds. Likewise, administration of arginine and α-ketoglutarate has also proven useful in treating ammonia intoxication and heightening liver detoxication in animal models. Not only was the survival rate found to be higher in the treatment group relative to the control, the treatment group also suffered fewer convulsive episodes.

In addition, arginine α-ketoglutarate has various uses as a source of α-ketoglutarate. By virtue of its role in the amino acid synthesis pathway, α-ketoglutarate exerts strong regulatory control over protein metabolism. Previous studies demonstrated its potency in conserving endogenous glutamine pools and increasing glutamine synthesis, which have particular benefits in clinical nutrition and metabolic care by countering trauma-induced catabolism. (Cynober (1999) Curr Opin Clin Nutr Metab Care, 2(1):33-7.) U.S. Pat. No. 5,646,187 describes the utility of α-ketoglutarate in treating critically ill patients for improving protein synthesis capacity, preserving lean body mass and maintaining energy status in skeletal muscle. Similarly, WO 89/03688 discloses the use of α-ketoglutarate to increase glutamine content in postoperative patients.

Alpha-ketoglutarate also possesses antioxidative properties, as supported by studies on hydrogen peroxide (H₂O₂)-induced hemolysis of human erythrocytes. The non-enzymatic oxidative decarboxylation of alpha-keto acids is shown to be involved in the hydrogen peroxide decomposition process. As part of the pathway leading to the citric acid cycle, α-ketoglutarate is crucial to energy generation. Studies in this area have yielded a significant correlation between leukocyte glutamate dehydrogenase deficiency and the presence of extrapyramidal signs, supranuclear palsy, absence of osteotendineal reflexes and neurogenic electromyographical findings. (Orsi et al. (1988) Acta Neurol Scand, 78(5):394-400.)

Furthermore, there is a significant link between genetic deficiency of glutamate dehydrogenase, an enzyme which converts glutamate to α-ketoglutarate, and certain dominantly inherited ataxias and olivopontocerebellar atrophy (OPCA). (Plaitakis et al. (1980) Ann Neurol, 7(4):297-303, Chokroverty et al. (1985) Neurology, 35(5):652-9.) Ataxia is a condition characterized by failure of motor control and/or irregularity of muscular action whereas OPCA refers to a group of ataxias characterized by progressive neurological degeneration affecting the cerebellum, the pons and the inferior olives.

In collagen synthesis, α-ketoglutarate plays an important role as one of the cofactors of prolyl hydroxylase and lysyl hydroxylase, enzymes responsible for hydroxylation of proline and lysine residues. Studies conducted on scorbutic animal models which characteristically exhibit lowered prolyl hydroxylase activity indicate that the enzyme activity could be increased by incubating homogenates with ascorbate (Vitamine C), ferrous ions, and α-ketoglutarate thereby alleviating the pathological symptoms. (Kuttan (1980) J Nutr, 110(8):1525-32.)

Alpha-ketoglutarate is also highly effective in preventing glycosylation/glycation of proteins associated with diabetic complications such as atherosclerosis, cataract formation, and retinopathy, and mere aging. Protein-bound advanced glycation endproducts (AGEs) can exert cytotoxic effects on neighboring cells and are, for example, the structural components of beta-amyloid plaques in Alzheimer's disease. Administration of α-ketoglutarate, however, attenuates the cytotoxicity of these AGEs via the compound's competitive inhibition of protein glycation and antioxidant properties. In the case of diabetic retinopathy, even careful monitoring of blood glucose levels does not necessarily preclude pathogenesis. Therefore, the intake of α-ketoglutarate is required in addition to a diabetic drug to prevent the glycation process in retinopathy.

### SUMMARY OF THE INVENTION

An oral formulation of two, three or four active ingredients is disclosed which formulation is comprised of these pharmaceutically active components with one or more excipient materials. A wide range of different formulations of the four main active ingredients in quick release as well as biphasic and controlled release formulations will be apparent to those skilled in the art upon reading this disclosure. The four active ingredients are (1) a lipid soluble thiamine, (2) lipoic acid, (3) a creatine derivative, and (4) L-arginine α-ketoglutarate. The formulation of the an excipient material is designed to obtain a desired result, e.g. (1) maintain sufficient blood levels of the thiamine to support nerve regeneration, (2) maintain sufficient blood levels of lipoic acid to reduce serum glucose levels, (3) provide sufficient levels of creatine derivative to improve muscle performance, and (4) provide arginine levels to improve circulation.

Formulations of the invention comprise two or more active components. One of the components may be a lipid soluble thiamine, e.g. benfotiamine or prosultiamine. One, two or more different lipid soluble thiamine compounds may be present together in the formulation or may be administered in separate oral formulations in the same treatment protocol of the same patient.

Another active component which may be present is lipoic acid which may be present as a racemic mixture, as the R-(+) enantiomer in amounts from 50% to 100% (of the lipoid acid component) or as the S-(−) enantiomer in amounts from 50% to 100% (of the lipoic acid component). If it is understood that if one enantiomer is present in an amount of more than 50% the other component is present in corresponding smaller percentage amounts. For example if the R-(+) enantiomer is present in amounts of 60%, 70%, 80%, 90% or 95% the S-(−) enantiomer is present in amounts of 40%, 30%, 20%, 10% or 5% respectively.

Another active component that may be present is a creatine derivative. The derivative may be a creatine ester such as the ethyl ester. Two, three or a plurality of creatine derivatives may be present in a single formulation.

Another active component that may be present is L-arginine α-ketoglutarate. Other forms (e.g. salts) of arginine may also be present in an oral formulation of the invention.

All four of these active components may be included in an oral formulation of the invention. Such formulations can be used for the treatment of patient, e.g. used to control blood glucose levels and treat diabetic polyneuropathy and other complications of diabetics including diabetic neuropathy, diabetic nephropathy, and macrovascular disease. The formulation of the invention makes it possible to obtain long term high plasma and tissue levels of lipid soluble thiamine. This allows for activation of the enzyme transketolase. When transketolase is activated, glucose is shunted into the pentose-phosphate pathway thereby reducing toxic effects of hyperglycemia. The formulation of lipoic acid and lipid-soluble thiamine provide a unique complimentary and synergestic combination of active ingredients for treating a wide variety of manifestation of diabetes arising from the toxicity of chronically elevated plasma glucose.

The creatine derivative (e.g. creatine ethyl ester) is useful in the treatment of human muscle tissue and improves muscle performance.

The arginine α-ketoglutarate improves circulation and as such enhances the delivery of other components of the formulation to the appropriate cells.

One aspect of the invention is a biphasic formulation which provides a quick release of a portion of the active components of the formulation followed by controlled release of the remainder which increases the period of time that a therapeutic level of the active components are continuously maintained in the patient. The therapeutic level as well as the period of time over which that level must be maintained can vary between patients based on a range of factors such as the condition of the patient and the patient's reactivity to the active components. However, an oral formulation of the invention can be formulated to maintain a therapeutic level over a period of time which is greater than that obtained with a conventional quick release formulation.

The ratio of active components to excipient material and the particular excipients used result in a formulation which allows the active components to be released quickly at first and thereafter in a controlled manner for absorption into the circulatory system. By maintaining a desired serum level of active components in blood serum the oral formulation of the invention achieves physiological effects which are superior to those obtained when higher serum levels are obtained for a short term with a quick release oral dosage formulation or a single dose injectable formulation.

By providing a biphasic formulation of active components the physiological effects are provided quickly at first to raise blood levels and then continually provided over a period of time resulting in improved nerve regeneration, reduced glucose levels and A1c levels, improve circulation, enhance muscle performance and thereby obtain a range of associated health benefits. The controlled release formulation of the invention shows that highly desirable therapeutic effects can be obtained by maintaining a therapeutic blood serum level of the active components over a period of time which is meaningfully longer than that obtained with a quick release formulation and results are improved by maintaining such day after day over a period of 3, 7, 10, 30, 60 or more days.

A formulation of the invention will preferably obtain initial levels of at substantially the same rate as a quick release formulation and thereafter maintain therapeutic levels over a period which is 10% or more, more preferably 50% or more and still more preferably 100% or more longer than a quick release formulation maintains therapeutic levels. To obtain a particularly preferred result the oral formulation of the invention will quickly release a sufficient amount of the active components so as to quickly obtain a therapeutic level and thereafter release the active components at a rate which substantially matches the rate at which each of the active componentss is being metabolized. Accordingly, a particularly preferred biphasic formulation is designed to (1) raise levels of the active components quickly to a therapeutic level; and (2) thereafter maintain a therapeutic level over a maximum amount of time based on the amount of active component in the formulation and to not significantly exceed the therapeutic level.

An aspect of the invention is an oral formulation of two, three, or four active components, and excipient compounds which is quick release, biphasic or controlled release.

Another aspect of the invention is a biphasic oral formulation of active component which provides an immediate release of a first portion of the formulation to quickly raise blood serum levels to a therapeutic level and a controlled release of a second portion to maintain a therapeutic level over a maximum amount of time.

An advantage of the method and formulation of the invention is that by maintaining relatively low serum levels of the active components over long periods of time (e.g. four hours or more per day) a range of desired results are obtained, e.g. serum glucose levels are suppressed over long periods thereby inhibiting adverse effects which result from abnormally high serum glucose levels.

Another advantage of the invention is that by administering the formulation over long periods the patient is provided with a reduced risk of developing insulin resistance and/or diabetes mellitus.

Another aspect of the invention is that the formulation provides a method of treating type 2 diabetes, i.e. non-insulin-dependent diabetes mellitus (NIDDM).

Yet another aspect of the invention is that the lipoic acid may be present as a racemic mixture or with the R-(+) enantiomer present in amounts greater than 50% and constituting up to 100% of lipoic acid in the formulation.

An advantage of the invention is that a convenient oral delivery dosage form is used to obtain the results which are superior to a single dose injectable.

Another advantage of the invention is that glucose levels can be reduced and be maintained at levels substantially below levels they were at prior to treatment via the present invention.

A feature of the invention is that the oral formulation may be a tablet, capsule, caplet, etc. containing any desired amount of the active components.

Another aspect of the invention is that it may be formulated with one or more additional active components such as antidiabetic agents e.g. sulfonylureas; biguanides and thiazolidinediones which agents may be formulated for quick release, controlled release or in a biphasic formulation.

Another aspect of the invention is a method of treatment whereby sustained low levels of active components in blood serum over long periods continually stimulate basal glucose transport.

Yet another aspect of the invention is the synergistic effect obtained by combining a plurality of active components together.

Still another aspect of the invention is that the effect of one component such as arginine improves circulation and as such improves circulation and as such improves the effects of the other active components.

Still yet another aspect of the invention is that the combined effects of the plurality of multiple components obtains results different from that obtained with any one of the components.

Another aspect of the invention is that the arginine and lipoic acid component both enhance the ability of the creatine derivative and lipid soluble thiamine components to reach their respective sites of action.

These and other objects, aspects, advantages, and features of the invention will become apparent to those persons skilled in the art upon reading the details of the invention as more fully described below.

### BRIEF DESCRIPTION OF THE DRAWING

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a conceptualized graph comparing a quick release oral dosage formulation to a biphasic lipoic acid oral dosage formulation wherein the amount released over time is graphed.

FIG. 2 is a graph of the percent of the particles versus sieve size for two different compositions of creatine derivatives.

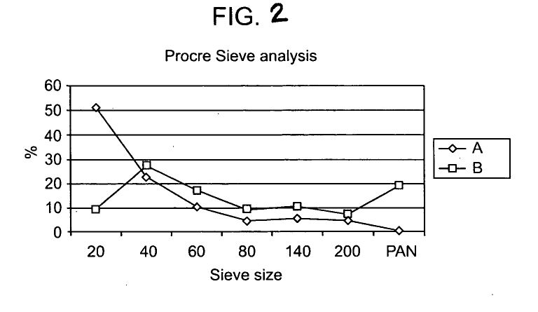

### DETAILED DESCRIPTION OF THE INVENTION

Before the present, formulations, methods and components used therein are disclosed and described, it is to be understood that this invention is not limited to particular compounds, excipients or formulations as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided are subject to change if it is found that the actual date of publication is different from that provided here.

### Definitions

The term “arginine α-ketoglutarate” is intended to mean arginine α-ketoglutarate which is a salt also known as arginine 2-ketoglutarate, arginine 2-oxoglutamate, and arginine 2-oxopentanedioic acid. Unless specified, the term covers the racemic mixture as well as any other (non-50/50) mixture of the enantiomers including substantially pure forms of either the R-(+) or the S-(−) enantiomer. Further, unless specified otherwise the term covers pharmaceutically acceptable salts (e.g. Na and K salts) and amides, esters and metabolites of the acid.

The terms “pharmaceutically acceptable salt” or “pharmaceutically acceptable salts” and the like are the terms is intended to encompass a conventional term of pharmaceutically acceptable acid addition salts which refer to salts which retain the biological effectiveness and properties of the free-base form of the acid and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malconic acid, succinic acid, maleic acid, fumaric, tartaric acid, citric acid, benzoic acid, cinnamic acid, mandelic acid, methanesulfonic acid, ethanesulfonic acid, p-toluenesulfonic acid, salicylic acid and the like. The same is true with respect to amides, esters and metabolites that is those forms which can be formed and maintain biological effectiveness and not have significant undesirable biological properties.

The term “lipoic acid” is intended to mean α-lipoic acid which is a chiral molecule also known as thioctic acid; 1,2-diethylene-3 pentanoic acid; 1,2-diethylene-3 valeric acid; and 6,8-thioctic acid. Unless specified the term covers the racemic mixture as well as any other (non-50/50) mixture of the enantiomers including substantially pure forms of either the R-(+) or the S-(−) enantiomer. Further, unless specified otherwise the term covers pharmaceutically acceptable salts (e.g. Na and K salts) and amides, esters and metabolites of the acid. The molecule formula is C₈H₁₄O₂S₂ the molecular weight is 206.32 and it has a pKa of 4.7. In referring to pharmaceutically acceptable salts the term is intended to encompass a conventional term of pharmaceutically acceptable acid addition salts which refer to salts which retain the biological effectiveness and properties of the free-base form of the acid and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malconic acid, succinic acid, maleic acid, fumaric, tartaric acid, citric acid, benzoic acid, cinnamic acid, mandelic acid, methanesulfonic acid, ethanesulfonic acid, p-toluenesulfonic acid, salicylic acid and the like. The same is true with respect to amides, esters and metabolites that is those forms which can be formed and maintain biological effectiveness and not have significant undesirable biological properties.

The term “creatine” refers to a compound having the following structural formula:

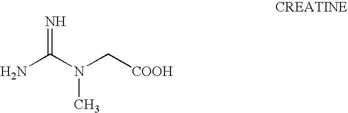

Further, unless specified otherwise the term covers pharmaceutically acceptable salts (e.g. Na and K salts) of the acid wherein the COOH is COONa. Thus, in the above structure the sodium salt is when COOH becomes COONa. In referring to pharmaceutically acceptable salts the term is intended to encompass a conventional term of pharmaceutically acceptable acid addition salts which refer to salts which retain the biological effectiveness and properties of the free-base form of the acid and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malconic acid, succinic acid, maleic acid, fumaric, tartaric acid, citric acid, benzoic acid, cinnamic acid, mandelic acid, methanesulfonic acid, ethanesulfonic acid, p-toluenesulfonic acid, salicylic acid and like forms which can be formed and maintain biological effectiveness and not have significant undesirable biological properties.

The term “creatinine” refers to a compound having the following structure:

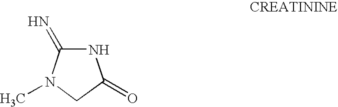

The term “excipient material” is intended to mean any compound forming a part of the formulation which is intended to act merely as a carrier, i.e., not intended to have biological activity itself beyond that of regulating release of a biologically active component.

The term “creatine derivative” refers to a compound having the following structure: 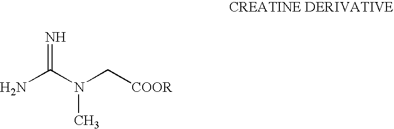
wherein R is not hydrogen but is hydrocarbyl.

The term “hydrocarbyl” is used herein to include substantially hydrocarbyl groups as well as purely hydrocarbyl groups. The description of these groups as being substantially hydrocarbyl means that they contain no non-hydrocarbyl substituents or noncarbon atoms which significantly affect the hydrocarbyl characteristics or properties of such groups relevant to their uses as described herein. Non-limiting examples of substituents which do not significantly alter the hydrocarbyl characteristics or properties of the general nature of the hydrocarbyl groups of this invention include the following: Alkyl including those comprising one to twenty carbons including lower alkyl e.g. methyl, ethyl, butyl, isobutyl, tertiary butyl, etc. Alkenyl including those comprising one to twenty carbons and lower alkenyl.

The term “lower” as used in the present specification and claims, when used in conjunction with terms such as alkyl, alkenyl, alkoxy, and the like, is intended to describe such groups which contain a total of up to 7 carbon atoms.

The term “chemical degradation” is intended to mean that an active component is subjected to a chemical reaction which disrupts its biological activity.

The term “particle size” refers to the size of particles of formulation of an active component and in particular a creatine derivative of the invention. The particle size is based on United States mesh size ranges. Mesh sizes are defined by the mesh size of sieves used to separate particles. Sieve sizes may be graduated and defined by the number of lines per inch of each sieve e.g. 50 lines per inch or 20 lines per inch. Size specifications are designated by organizations such as ANSI and FEPA. Indicating a size of 30/40 U.S. mesh means that most of the particles in the formulation would fall between 30 mesh and the 40 mesh sieve. Standards permit a small amount of oversize and undersize materials. However, the undersized materials generally range to 2 to 4% as do the oversize materials. In formulating a creatine derivative active component into a formulation of the invention it has been found that a formulation which is processed so that the particles or creatine would fall between a sieve 18 and sieve 60, or a sieve 20 and a sieve 40 can be made flowable and the flowable material can be compressable into a tablet in accordance with the invention. A sieve 18 has a sieve opening of 1,000 microns, sieve 20 has an opening of 841 microns; sieve 25 has an opening of 707 microns; sieve 30 has an opening of 595 microns; sieve 35 has an opening of 500 microns; sieve 40 has an opening of 420 microns; sieve 45 has an opening of 354 microns; sieve 50 has an opening of 297 microns; sieve 60 has an opening of 250 microns.

The terms “treating” and “treatment” and the like are used herein to generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease and/or its symptoms or conditions. The invention is directed towards treating using two or more active components in a single oral dosage unit. Depending on the active components it may include a creatine derivative and result in enhancement of muscle performance, building muscle tissue, treating a neuromuscular disorder, improving muscle endurance or reducing fat tissue. Formulations of the invention, comprised of a creatine derivative may be administered to patients having myoclonus (i.e., a neuromuscular disorder characterized by the occurrence of irregular, asynergic, and jactitious contractions of muscles producing non repetitive, brief, involuntary movements in various body areas) as a symptom of epilepsy, neurodegenerative disease such as Parkinson's disease, multiple sclerosis or amyotrophic lateral sclerosis (ALS) and Tourette's syndrome. When an arginine component is present the results may be improved circulation. The formulation may be directed towards treating patient's symptoms from glutamate dehydrogenase deficiency and depressed prolyl hydroxylase and lysyl hydroxylase activity. The present invention is involved in preventing, inhibiting, or relieving adverse effects attributed to glycation of proteins characteristics of antherosclerosis, cataract formation, retinopathy, and aging. When lipoic acid and/or a lipid soluble thiamine is present the method of treatment of the invention may be directed towards treating patient's suffering from a disease related to diabetes mellitus including adverse effects due to abnormally high levels of glucose as well as diabetic polyneuropathy and the effects of free radicals and/or oxidizing agents over long periods of time. The present invention is involved in preventing, inhibiting, or relieving adverse effects attributed to abnormally elevated serum glucose levels over long periods of time and/or are such caused by free radicals or oxidizing agents present in a biological system over long periods of time.

The terms “synergistic”, “synergistic effect” and the like are used interchangeably herein to describe improved treatment effects obtained by combining two or more of (1) lipoic acid, (2) a lipid soluble thiamine, (3) a creatine derivative, and (4) an arginine such as arginine α-ketoglutarate. Although a synergistic effect in some fields means an effect which is more than additive (e.g., one plus one equals three) in the field of treating diabetes, circulation and muscles and related field an additive (one plus one equals two) or less than additive (one plus one equals 1.3) effect may be synergistic. For example, if a patient has an abnormally high glucose level, e.g. 400 mg/dl, that patient's glucose level might be reduced to 300 mg/dl by a conventional orally effective antidiabetic compound. Further, at a different time the same patient with a glucose level of 400 mg/dl might be administered a different orally effective antidiabetic compound which compound reduced the patient's glucose levels from 400 to 300 mg/dl. However, if both orally effective antidiabetic compounds are administered to the patient one would not ordinarily expect an additive effect thereby obtaining a reduction to 200 mg/dl and may obtain no more of a reduction in glucose level than when either drug is administered by itself. Further, combinations of two or more compounds may have an effect which is less than either compound alone. If additive effects could always be obtained then diabetes could be readily treated in all instances by coadministering several different types of orally effective antidiabetic compounds until the disease is cured—but this approach is not an effective treatment. In a similar manner circulation and muscle performance could be treated by adding multiple drugs together. Such is not effective in general. However, in connection with the present invention coadministration of formulations of two or more active components is better than one component. For example, administering controlled release lipoic acid with a lipid soluble thiamine will obtain results which are synergistic, i.e. greater than the effects obtained by the administration of either composition by itself. These two active compounds may be further administered with one or more additional orally effective antidiabetic compounds such as metformin hydrochloride to obtain a further enhanced or “synergistic” result which may be less than additive but more than either by itself.

The term “quick release formulation” refers to a conventional oral dosage formulation. Such a formulation may be a tablet, capsule, pill, liquid suspension or the like designed to provide for substantially immediate release of the active ingredient and includes enteric coated oral formulations which provide some initial protection to the active ingredient and thereafter allow substantially immediate release of substantially all the active ingredient. A quick release formulation is not formulated in a manner so as to obtain a gradual, slow, or controlled release of the active ingredient.

The terms “biphasic formulation,” “biphasic dosage form” and the like are used interchangeably here to describe any oral formulation with two different release rates. As an example, the biphasic formulation provides for an immediate release of a first portion of all or any of the active components followed by a slower, controlled and metered release of a second portion of the remainder of all or any of the active components. Thus, a biphasic formulation of the invention preferably quickly raises blood levels to a therapeutic level of the desired active components and thereafter provides for a slower release which maintains the therapeutic level over a substantially longer time as compared to a quick release (10%, 50%, 100% or 200% longer) preferably without significantly exceeding the therapeutic level.

“Thimiane” is also referred to as vitamin B₁ and is C₁₂, H₁₇, ON₄, S HCI or thiamine hydrochloride. The compound is soluble in water and insoluble in ether and lipids. The RDA for vitamin B1 is about 1.2 mg. per day, or 1.4 mg. during pregnancy or lactation. Infants need more per body weight though less in total, about 0.5 mg. per day. Thiamine needs are based on many factors; given good health, we need about 0.5 mg. per 1,000 calories consumed, since B1 is required for energy metabolism. So our needs are based on body weight, calorie consumption, and the amount of vitamin B1 synthesized by intestinal bacteria, which can vary greatly from person to person.

Thiamine is a coenzyme for the decarboxylation of pyruvate and the oxidation of alpha keto-glutamic acid. Lipoic acid which is formed in the liver is also required for the reactions. Patients with liver disease may show signs of B1 deficiency, possibly because of deficient synthesis of lipoic acid. In vitro, thiamine deficiency produces accumulation of pyruvate and lactate, reduction of acetate, citrate and alpha-keto-glutarate and reduced acetylcholine synthesis. Any of these metabolic changes could be involved in dysfunction.

The term “lipid soluble thiamine” is used here to cover derivatives of thiamine with higher solubility in lipids as compared to thiamine., e.g. 10%, 50%, 100%, 200% or 10 times or more, more soluble in lipids as compared to thiamine. Specific lipid soluble thiamines include benfotiamine and prosultiamine. The term as used here is intended to cover pharmaceutically acceptable salts, acids, and esters thereof.

### Formulation in General

A plurality of active components are included in an oral dosage unit. The component present may be all of or any two or three of (1) a lipid soluble thiamine, (2) lipoic acid, (3) a creatine derivative, and (4) α-ketoglutarate. The amount of each component present can vary. The lipid soluble thiamine is generally present in small amounts such as 0.5, 1.0 or 5 mg. However, in some formulation the amount can range from 0.5 mg to 500 mg with amounts of 10, 50, 100 and 200 mg being useful. The lipoic acid component may be present in amounts of from 25 mg to 600 mg with amounts of 50, 100, 200, 300 and 400 mg being useful. The creatine derivative which may be creatine ethyl ester may be present in amounts from about 100 mg to 2,000 mg with 100 mg increments (i.e. 200 mg, 300 mg, etc.) being useful. The arginine α-ketoglutarate component may be present in amounts of from 100 to 1,000 mg with 100 mg increments (i.e. 200 mg, 300 mg, etc.) being useful.

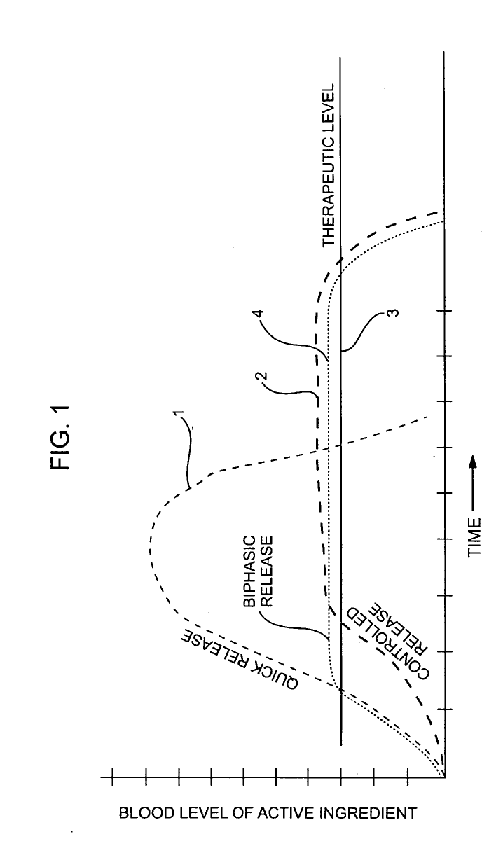

Referring to FIG. 1 which is a conceptualized graph provided to show a comparison between a theoretical quick release and theoretical biphasic oral formulation. The graph shows the amount of the active components in the patient over time. The light dashed line 1 is of a theoretical quick release oral formulation showing that the level of active component rises and falls quickly. The bold dashed line 2 is of a theoretical controlled release formulation which initially rises more slowly as compared to the quick release formulation after reaching the therapeutical level shown by the solid line 3 it enters the controlled release phase and maintains a level at or just above the therapeutic level until no more active component is available in the dosage form. At this point the line drops to zero quickly as there is no more active component in the formulation for release and remaining active component is metabolized.

The dotted line 4 shows the release rate of a biphasic formulation. In the first phase, release rate of the active component is substantially the same as the quick release formulation. The biphasic formulation reaches the therapeutic level at substantially the same time as the quick release formulation does. Thereafter, the biphasic formulation begins a slower release as compared to the quick release formulation. For example, the rate of release of active component in the second phase is substantially equal to the rate at which the active components are metabolized. As with the controlled release formulation the object is to keep the level as close to the therapeutic level as possible for as long as possible.

In one aspect of the invention each of the individual active components are separately formulated with excipient and thereafter combined. This is done, for example, because components such as lipoic acid is metabolized more quickly as compared to lipid soluble thiamines. In one embodiment the lipid soluble thiamine creatine derivative and arginine α-ketoglutarate are all in a quick release formulation and combined with controlled release lipoic acid in a biphasic formulation, i.e. both quick release and controlled release in a single formulation. Such a formulation obtains enhanced bioavailability of a thiamine by using a lipid soluble molecule and improved bioavailability of creatine by using a creatine derivative and increases the length of time that therapeutic levels of lipoic acid are maintained via the biphasic controlled release formulation of that component.

The formulation of the invention is preferably an oral dosage formulation which may be in any suitable oral form including tablets, pills, capsules, caplets, liquid suspensions, etc. The dosage may be of any desired size in terms of the active ingredients. However, sizes for the combined active ingredients in a range of about 200 mg to about 5,000 mg are generally used, or for example 400 mg to 2,000 mg or alternatively about 500 mg to about 1,000 mg.

These amounts of the different active components can be total amounts per day or can be modified to be amounts per day per 1,000 calories consumed by the patient.

The biphasic formulation is constructed to hold the active components in different combinations of excipients. Preferably the center portion of the formulation will be produced in accordance with the examples provided here. The outer portion of the formulation could be the active components alone or mixed with any excipients in the same proportional amounts generally used by those of ordinary skill in the art in producing a conventional quick release formulation.

The quick release portion may comprise from about 10% to about 50% of the active components in the formulation or preferably about 20% to about 30% and more preferably about 25% of the active components in the formulation.

The amount a patient will need to obtain an optimum therapeutical effect will vary with a number of factors known to those skilled in the art e.g. the size, age, weight, sex and condition of the patient. The patient may begin with daily doses of about 300 mg of lipoic acid, 50 mg of benfotiamine, 300 mg of arginine α-ketoglutarate and 600 mg of creatine ethyl ester, and determine if desired results are obtained, e.g. glucose levels are reduced to acceptable levels and muscle performance improved. If the desired results are not obtained in one week the daily dosage amount can be increased in increments of 25, 50, or 100 mg or doubling the dose or more of the active components. For example, lipoic acid increases can be in amounts of 100 to 300 mg/day up to any useful amount e.g. 2,000 mg/day. Longer time periods such as 3 month, 6 months, 12 months or longer may be required to observe improved results in other areas such as decreases in diabetic polyneuropathy.

The active components may be administered in multiple tablets, capsules etc. to reduce the size of any one. For example, a suggested dosage is to administer two tablets in the morning and administer one tablet four hours later and repeat daily over five or more days where the tablets comprise 300 mg of lipoic acid and 50 mg of benfotiamine 300 mg of arginine α-ketoglutarate and 600 mg of creatine ethyl ester. The larger initial dosage in the morning is effective in obtaining a desired effect which after being obtained can be maintained by a lower dose. Thus, a biological system may be “kick started” by a high therapeutic level and then maintained at a lower level which is also therapeutic in terms of obtaining a desired result. In a particularly preferred formulation the benfotiamine is present as 50 mg of quick release and 75 mg of the 300 mg of lipoic acid is in a quick release formulation in the outer shell of the tablet and the inner 225 mg is in a controlled release formulation. The arginine α-ketoglutarate and creatine ethyl ester may both be in the quick release form.

### Lipoic Acid

The manufactured compound α-lipoic generally exists as a 50/50 or racemic mixture of R-(+)-α-lipoic acid and S-(−)-α-lipoic acid. The R-(+) enantiomer is the naturally produced biological form of the compound and as such is believed to be largely responsible for obtaining the physiological effect of the lipoic acid component. Thus, the lipoic acid ingredient of the formulation of the present invention may be 100% R-(+) enantiomer. However, the active ingredient may be present in any mix of the two enantiomers e.g. 10% S-(−) and 90% R-(+); 25% S-(−) and 75% R-(+). Further, it should be noted that even though the R-(+) enantiomer is believed to be the more active the S-(−) enantiomer may possess unique properties which make inclusion of the S-(−) enantiomer important in any formulation used in treatment. Unless stated otherwise information disclosed here refers to formulations containing a racemic mixture. If the active ingredient is not a racemic mixture then some adjustment may be needed in the formulation in order to account for the greater activity of the R-(+) enantiomer as well as the slightly longer half life of the R-(+) enantiomer compared to the S-(−) enantiomer.

A typical controlled release formulation or portion of the formulation may contain about 50-70% by weight active ingredient with the remainder being excipient material. The quick release portion of the formulation may comprise 100% active components or a very small amount e.g. 5-10% by weight of excipient. The controlled release portion of the formulation may comprise 55% to 65% active ingredient and more preferably about 60% active ingredient by weight. Thus, an oral formulation of the invention may comprise about 300 mg of lipoic acid, 50 mg of benfotiamine or prosultiamine 300 mg arginine α-ketoglutarate, 600 mg of creatine ethyl ester and about 200 mg of excipient material. Human patients generally eat during the day and sleep at night. Eating causes increased glucose levels. Accordingly, it is generally preferable to give a larger dose of lipoic acid at the beginning of the day. This may include two of the tablets. Later in the day (about 4 hours) the patient will take an additional tablet for a typical daily dose of about 900 mg of lipoic acid and 150 mg of benfotiamine, 300 mg of arginine α-ketoglutarate and 1,800 mg of creatine ethyl ester, for a 70 kg man—all amounts ±20% or ±10%.

The formulation may be produced so that it is characterized by (a) protecting the active ingredient (to the extent required) from chemical degradation in a patient's gastrointestinal tract and (b) releasing the active ingredient in a controlled manner. By gradually releasing the active ingredient the serum levels of the active components obtained are (1) lower than those obtained with single dose injectable or a non-controlled release formulation; and (2) maintained over longer periods of time than obtained with single dose injectable or a non-controlled release formulation. A preferred biphasic formulation of the invention releases active ingredient so as to obtain a blood serum level in a human patient in a range of about 25 to 2,500 ng/ml of plasma for lipoic acid; 5 to 500 ng/ml of plasma for benfotiamine 35 to 65 ng/ml of plasma for arginine α-ketoglutarate, and 50 to 300 micrograms/ml of plasma for the creatine component. The range is preferably about 50 to 2,000 ng/ml of plasma and more preferably about 1,800 ng/ml of plasma for lipoic acid; 10 to 400 ng/ml of plasma for lipid soluble thiamine 50 ng/ml for the arginine and 75 to 125 micrograms/ml for the creatine component, with all numbers being ±20% or ±10% or ±5%. The plasma level that is therapeutic will vary somewhat from patient to patient depending on factors such as the weight, sex and age and condition of the patient and will vary further depending on the therapy or treatment being sought.

Some characteristics of lipoic acid are (1) it is non-toxic at relatively high levels, i.e. levels well in excess of therapeutic levels; and (2) lipoic acid is quickly metabolized by human patients. The present invention relies in part on the discovery that lipoic acid provides desirable therapeutic results even at very low levels provided those low levels are maintained over an extended period of time whereas therapeutic results are not obtained (even with higher levels) if the therapeutic level is not maintained over a sufficiently long period of time. Further, the present invention relies in part on the discovery that therapeutic results are further improved if the delivery of lipoic acid is administered over a period of five or more, preferably thirty or more consecutive days with long periods (four hours, eight hours, or 12 hours or more) of therapeutic levels of lipoic acid being obtained on each of the days. Another aspect of the invention is the synergistic effect obtained by comfirming the effects of lipoic acid with other active ingredients. Yet another aspect of the invention is the improved bioavailability of both a creatine ester as compared to creatine and of a lipid soluble thiamine as compared to a water soluble thiamine.

One aspect of the invention is that a range of highly desirable therapeutic effects are obtained even when the lipoic acid blood serum levels are maintained in a range well below those used in other formulations. The present invention could obtain desired therapeutic effects with higher levels of lipoic acid in blood serum. However, at least minimum levels should be constantly maintained over a long period of time (4 hours or more per day) for a plurality of days to obtain the desired results. When the oral dosage form is designed to obtain the lowest possible therapeutic level over the longest possible time period the results obtained are maximized and the amount of drug needed is minimized.

The lipoic acid blood plasma level obtained via the present invention may be insufficient to obtain a desired therapeutic effect if that level is maintained for only a short period of time. The amount of time and the level needed can vary based on factors such as the condition of the patient and the results desired. In general, longer periods at a sustained level are preferred to short periods and large fluctuation in levels. By using the biphasic oral formulation of the invention therapeutic lipoic acid blood plasma levels can be maintained over 8 hours or more, preferably over 12 hours or more and more preferably over 16 hours or more per day. Further, those lipoic acid blood plasma levels over these periods of time are repeatedly obtained on consecutive days, preferably for weeks or months and more preferably continuously over any period during which the patient would benefit from reduced serum glucose levels—which may be the remainder of the patient's life.

To obtain the desired results a formulation of the invention needs to start with a sufficient amount of lipoic acid such that it is capable of releasing enough lipoic acid per unit of time to obtain the desired lipoic acid serum levels while compensating for lipoic acid which is metabolized. To obtain the desired results the biphasic formulation provides an initial release of lipoic acid quickly and thereafter provides a gradual release which slows over the useful life of the formulation. Desired results can be obtained with a single phase controlled release formulations where the release may be gradual from the beginning. In either case there is preferably a gradual slowing of the rate of release which is compensated for in that some of the previously released lipoic acid remains in the blood serum unmetabolized.

A preferred oral formulation is a tablet which is designed to provide an initial quick release of a portion of the lipoic acid, e.g. about 25% and thereafter dissolve gradually over a period of about 8 hours. As the tablet dissolves its reduced size will release smaller and smaller amounts of lipoic acid per unit of time. However, because the individual's system already contains a therapeutic level of lipoic acid the slower release rate is sufficient to match the rate of lipoic acid being metabolized and such will result in maintaining a relatively constant therapeutic level as shown in FIG. 1. At the end of the time when release of lipoic acid is no longer taking place (e.g. about 4 to 8 hours) another tablet is administered and the process is repeated. To obtain the benefits of the invention the process is continually repeated over a plurality of consecutive days, for weeks, or for months or for years. By maintaining a minimal lipoic acid blood serum level over time a patient's abnormally high serum glucose levels are reduced and the long term adverse effects of elevated serum glucose levels are avoided.

### Creatine Derivative

Creatine ethyl ester is characterized as (1) non-toxic at relatively high levels, i.e., levels well in excess of therapeutic levels; and (2) metabolized by human patients to the same metabolites as creatine. The present invention relies in part on the discovery that creatine esters provide desirable therapeutic results even at very low levels provided those low levels are maintained over an extended period of time; whereas therapeutic results are not obtained (even with higher levels) if the therapeutic level is not maintained over a sufficient period of time. Further, the present invention relies in part on the discovery that therapeutic results are further improved if the formulation is delivered over a period of five or more days, preferably thirty or more consecutive days with long periods of therapeutic levels of creatine being obtained on each of the days.

### Arginine α-Ketoglutarate

Arginine α-ketoglutarate is characterized as (1) non-toxic at relatively high levels, i.e., levels well in excess of therapeutic levels; and (2) quickly metabolized by human patients. The present invention relies in part on the discovery that arginine α-ketoglutarate provides desirable therapeutic results even at very low levels provided those low levels are maintained over an extended period of time; whereas therapeutic results are not obtained (even with higher levels) if the therapeutic level is not maintained over a sufficient period of time. Further, the present invention relies in part on the discovery that therapeutic results are further improved if the formulation is delivered over a period of five or more days, preferably thirty or more consecutive days with long periods of therapeutic levels of arginine α-ketoglutarate being obtained on each of the days.

One aspect of the invention is that a range of highly desirable therapeutic effects are obtained even when the arginine α-ketoglutarate blood serum levels are maintained in a range well below those previous used. The present invention could obtain desired therapeutics effects with higher levels of arginine α-ketoglutarate in blood serum. However, at least minimum levels would need to be constantly maintained over a long period of time (4 hours or more per day) for a plurality of days to obtain the desired results. When the oral dosage form is designed to obtain the lowest possible therapeutic level over the longest possible time period the results obtained are maximized and the amount of drug needed is minimized.

The arginine α-ketoglutarate blood plasma level obtained via the present invention is insufficient to obtain a desired therapeutic effect if that level is maintained for only a short period of time, e.g., 4 hours or less. However, by using the controlled release formulation of the invention these lower arginine α-ketoglutarate blood plasma levels can be maintained over 8 hours or more, preferably over 12 hours or more and more preferably over 16 hours or more per day. Further, those arginine α-ketoglutarate blood plasma levels over these periods of time are repeatedly obtained over a period of days, preferably weeks or months and more preferably continuously over any period during which the patient would benefit from, for example, the substance's inhibition of protein glycation—which may be the remainder of the patient's life.

To obtain the desired results, a formulation of the invention includes a sufficient amount of arginine α-ketoglutarate such that it is capable of releasing enough arginine α-ketoglutarate per unit of time to obtain the desired arginine α-ketoglutarate serum levels while compensating for arginine α-ketoglutarate which is metabolized. To obtain the desired results the formulation may immediately and quickly provide an initial release of arginine α-ketoglutarate and thereafter provide a gradual release which slows over the useful life of the formulation. However, the release may be gradual from the beginning. In either case, there is a gradual slowing of the rate of release which is compensated for in that some of the previously released arginine α-ketoglutarate remains in the blood serum unmetabolized.

A preferred oral formulation is a tablet which is designed to dissolve gradually over a period of about 8 hours. As the tablet dissolves, its reduced size will release smaller and smaller amounts of arginine α-ketoglutarate per unit of time. However, because the individuals system already contains a therapeutic level of arginine α-ketoglutarate, the slower release rate is sufficient to match the rate of arginine α-ketoglutarate being metabolized and such will result in maintaining a relatively constant therapeutic level. At the end of the time when release of arginine α-ketoglutarate is no longer taking place (e.g., about 4 to 8 hours), another tablet is administered and the process is repeated. To obtain the benefits of the invention, the process is continually repeated over a plurality of days, weeks, months or years. By maintaining a minimal arginine α-ketoglutarate blood serum level over time, a patient's symptoms arising from a genetic glutamate dehydrogenase deficiency and depressed prolyl hydroxylase and lysyl hydroxylase activity would be alleviated. Likewise, those patients at increased risk for atherosclerosis, cataract formation, and retinopathy would have lowered their chances of pathogenesis.

### Combination Formulations

Lipoic acid acts directly on muscle cells to stimulate glucose transport. The effect on serum glucose reduction obtained with lipoic acid may be sufficient for some patients. However, if an insufficient glucose lowering effect results the lipoic acid may be supplemental with one or more orally effective antidiabetic agents selected from the group consisting of sulfonylureas, biguanides and thiazolidiones. Useful sulfonylureas include tolbutamide and glipizide and related compounds such as Amaryl, Pandin and Starlix. These drugs target pancreatic beta cells and stimulate these cells to release insulin. The biguanides include compounds such as metformin, phenformin and buformin. These compounds act on the liver to decrease hepatic glucose output and on the intestine to block glucose uptake into the blood. Thiazolidinediones include compounds such rosiglitazone and pioglitazone. These compounds are believed to sensitize muscle and fat cells to insulin.

Although all or any orally effective antidiabetics can be formulated with or administered along with the formulation of the invention it is preferable to administer metformin (particularly metformin Hydrochloride tablets sold as Glucophage™) with controlled release formulations of the invention comprising therapeutically effective amounts of both lipoic acid and a lipid soluble thaimine. Some particularly preferred formulations include 300 mg lipoic acid (racemic or R(+) α lipoic acid), 50 mg lipid soluble thiamine (benfotiamine or prosultiamine) and 500 mg of metformin hydrochloride or if a larger dose is needed 600 mg of lipoic acid, 100 mg of lipid soluble thiamine and 1,000 mg of metformin hydrochloride. Additional enhanced effects may be obtained by taking a formulation of the invention along with vitamin C and/or vitamin E. For example a patient might take 900 mg/day of lipoic acid 50 to 100 mg/day of benfotiamine, 1,000 to 3,000 mg/day of vitamin C and 400 to 800 mg/day of vitamin E.

Example 10 provides specific examples of patient's which underwent coadministration of controlled release lipoic acid formulations of the present invention in combination with other treatments conventionally used to lower serum glucose levels. The synergistic effects were obtained, i.e. the combination of lipoic acid controlled release formulations of the invention with other therapeutic agents obtained results which were greater than results which might be expected with the administration of either composition by itself. The lipid soluble thiamine and optional antidiabetic component may be (1) solely in the quick release portion of the formulation; (2) solely in the controlled release portion of the formulation; or (3) in both portions of the biphasic formulation with any amount in either phase of the formulation.

### Excipient Material

Examples provided here show that formulations of the invention may comprise different amounts and ratios of active ingredient and excipient material. Further, different excipients can be used. Particularly preferred excipients and amounts used are recited in the Examples. However, upon reading the disclosure those skilled in the art will come to understand the general concepts of the invention and will recognize that other excipients, amounts, ratios and combinations might be used to obtain the results first shown here.

Although multiple doses of an oral formulation could be taken it is preferable to design the dosage such that a single dose is taken at each dosing event—preferably three times a day and more preferably twice a day. The better the active ingredient is protected from degradation the less active ingredient is needed in the original dosage thereby reducing manufacturing costs and increasing profits. The formulation must protect at least as much of the dose as is needed to obtain a pharmacological effect and preferably obtain the desired treatment results, e.g. maintaining desired lipoic acid and thiamine serum levels needed to obtain therapeutic results, e.g.,a reduced serum glucose level over time.

The formulation may be designed so that it releases the active ingredients gradually over time at a controlled rate of release which rate is preferably constant over 4 hours or more. This is particularly important for the lipoic acid component because (1) lipoic acid has a relatively short half life and (2) a desired level of lipoic acid in blood serum must be maintained over a long period to obtain the desired effect. If all of the lipoic acid is released at once it will all enter the circulatory system at once and be metabolized in the liver thereby causing the lipoic acid serum level to drop below the desired level. When this occurs the effect on reducing glucose levels is suboptimal.

### Typical Two Component Formulations

A typical two active component formulation of the invention will contain about 50% to 70% by weight of lipoic acid and 5% to 15% of lipid soluble thiamine and a particularly preferred formulation will comprise 60% by weight of lipoic acid and 10% lipid soluble thiamine. Assuming a formulation with 60% by weight of lipoic acid 10% by weight of lipid soluble thiamine with the remaining 30% being excipient material there are a number of possible components which could be used to make up that 30%. A generalized and specific description of such is provided below:

| | | |
|---|---|---|
| (1) | lipoic acid | 60% |
| | lipid soluble thiamine | 10% |
| | organic polymer | 30% |
| | TOTAL | 100% |
| (2) | lipoic acid | 60% |
| | lipid soluble thiamine | 10% |
| | organic polymer | 24.5% |
| | Inorganics | 5.5% |
| | TOTAL | 100% |
| (3) | lipoic acid | 60% |
| | lipid soluble thiamine | 10% |
| | organic polymer | 20%-30% |
| | Inorganics | 10% |
| | | or less |
| | TOTAL | 100% |
| (4) | lipoic acid | 60% |
| | lipid soluble thiamine | 10% |
| | microcrystalline cellulose | 9% |
| | cellulose acetate phthalate aqueous dispersion | 10% |
| | Polyvinylpyraolidone | 3% |
| | ethyl acetate | 2.5% |
| | hydrous magnesium silicate (talc) | 1% |
| | carboxy methyl ether | 4% |
| | magnesium stearate | 0.5% |
| | TOTAL | 100% |
| (5) | lipoic acid | 60% |
| | lipid soluble thiamine | 10% |
| | microcrystalline cellulose | 10-20% |
| | cellulose acetate phthalate aqueous dispersion | 5-15% |
| | Polyvinylpyraolidone | 1-5% |
| | ethyl acetate | 1-5% |
| | hydrous magnesium silicate (talc) | 0.5-3% |
| | carboxy methyl ether | 1-5% |
| | magnesium stearate | 0.5-1.5% |
| | TOTAL | 100% |
| (6) | R-(+)-α- lipoic acid | 60% |
| | Benfotiamine | 10% |
| | microcrystalline cellulose, NF (Avicel PH 101) | 9% |
| | Aquacoat CPD-30 (30% solids w/w) | 10% |
| | Plasdone K29/32, USP | 3% |
| | Carbopol 974P, NF | 2.5% |
| | Talc, USP | 1.0% |
| | croscarmellose sodium, NF (Ac, di-Sol) | 4.0% |
| | Magnesium Stearate, NF | 0.5% |
| | TOTAL | 100% |
| (7) | R-(+)- α - lipoic acid | 60% |
| | Prosultiamine | 10% |
| | microcrystalline cellulose, NF (Avicel PH 101) | 10-20% |
| | Aquacoat CPD-30 (30% solids w/w) | 5-15% |
| | Plasdone K29/32, USP | 1-5% |
| | Carbopol 974P, NF | 1-5% |
| | Talc, USP | 0.5-3% |
| | croscarmellose sodium, NF (Ac, di-Sol) | 1-5% |
| | Magnesium Stearate, NF | 0.5-1.5% |
| | TOTAL | 100% |

In the above formulation either or both of the lipoic acid and lipid soluble thiamine can be replaced with either or both of a creatine derivative (e.g. creatine ethyl ester) and arginine α-ketoglutarate.

### Three Component Formulations

A typical three component formulation of the invention will contain about 20% to about 50% by weight of each of the three active components and a particularly preferred formulation will comprise 35% ±10% by weight of each of the three active components. Assuming a formulation with about 35% ±10% by weight of each of the three active components with the remaining being excipient material, there are a number of possible components which could be used to make up the remainder of the formulation. A generalized and specific description of such is provided below:

| | | |
|---|---|---|
| (1) | Creatine ester | 35% |
| | Arginine α-ketoglutarate | 25% |
| | Lipoic Acid | 20% |
| | biodegradable polymer | 20% |
| | TOTAL | 100% |
| (2) | Creatine ester | 35% |
| | Arginine α-ketoglutarate | 20% |
| | Lipoic Acid | 25% |
| | biodegradable polymer | 14.5% |
| | Inorganics | 5.5% |
| | TOTAL | 100% |
| (3) | creatine ester | 30% |
| | Arginine α-ketoglutarate | 25% |
| | Lipoic Acid | 25% |
| | organic polymer | 10%-20% |
| | Inorganics | 10% |
| | | or less |
| | TOTAL | 100% |
| (4) | creatine ester | 25% |
| | Arginine α-ketoglutarate | 35% |
| | Lipoic Acid | 20% |
| | microcrystalline cellulose | 4% |
| | Cellulose acetate phthalate aqueous | 5% |
| | dispersion | |
| | Polyvinylpyrolidone | 3% |
| | ethyl acetate | 2.5% |
| | hydrous magnesium silicate (talc) | 1% |
| | carboxy methyl ether | 4% |
| | magnesium stearate | 0.5% |
| | TOTAL | 100% |
| (5) | creatine ester | 40% |
| | Arginine α-ketoglutarate | 20% |
| | Lipoic Acid | 20% |
| | microcrystalline cellulose | 5-20% |
| | Cellulose acetate phthalate aqueous | 5-15% |
| | dispersion | |
| | Polyvinylpyrolidone | 1-5% |
| | ethyl acetate | 1-5% |
| | hydrous magnesium silicate (talc) | 0.5-3% |
| | carboxy methyl ether | 1-5% |
| | magnesium stearate | 0.5-1.5% |
| | TOTAL | 100% |
| (6) | creatine ester | 30% |
| | Arginine α-ketoglutarate | 20% |
| | Lipoic Acid | 20% |
| | microcrystalline cellulose, NF | 14% |
| | (Avicel PH 101) | |
| | Aquacoat CPD-30 (30% solids w/w) | 5% |
| | Plasdone K29/32, USP | 3% |
| | Carbopol 974P, NF | 2.5% |
| | Talc, USP | 1.0% |
| | croscarmellose sodium, NF (Ac, di-Sol) | 4.0% |
| | Magnesium Stearate, NF | 0.5% |
| | TOTAL | 100% |
| (7) | creatine ethyl ester | 35%-40% |
| | Arginine α-ketoglutarate | 20-22.5% |
| | Lipoic Acid | 20-22.5% |
| | Diacalcium phosphate | 5-15% |
| | polyvinyl pyrrolidone | 2-4% |
| | Starch | 2-4% |
| | Magnesium Stearate, NF | 0.5-1.5% |
| | TOTAL | 100% |
| (8) | creatine ethyl ester | 33% |
| | Arginine α-ketoglutarate | 25% |
| | Lipoic Acid | 25% |
| | Di-Calcium Phosphate | 10% |
| | polyvinyl pyrrolidone (Kollidon 90) | 3% |
| | Starch | 3% |
| | Magnesium stearate | 1% |
| | TOTAL | 100% |
| (9) | creatine ethyl ester | 50% |
| | Arginine α-ketoglutarate | 15% |
| | Lipoic Acid | 15% |
| | Poly-DL-lactide-co-glycolide (PLG) | 20% |
| | TOTAL | 100% |
| (10) | creatine ethyl ester | 30% |
| | Arginine α-ketoglutarate | 20% |
| | Lipoic Acid | 20% |
| | hydroxypropyl methylcellulose | 20% |
| | Spray-dried lactose | 9.5% |
| | Magnesium stearate | 0.5% |
| | TOTAL | 100% |
| (11) | creatine ethyl ester | 30-35% |
| | Arginine α-ketoglutarate | 20-22.5% |
| | Lipoic Acid | 20-22.5% |
| | polyvinyl pyrrolidone (Kollidon 90) | 10-20% |
| | Lactose | 5-15% |
| | microcrystalline cellulose | 4-6% |
| | titanium dioxide | 1-5% |
| | TOTAL | 100% |
| (12) | creatine ethyl ester | 40% |
| | Arginine α-ketoglutarate | 20% |
| | Lipoic Acid | 20% |
| | polyvinyl pyrrolidone (Kollidon 90) | 20% |
| | TOTAL | 100% |
| (13) | creatine ethyl ester | 40% |
| | Arginine α-ketoglutarate | 25% |
| | Lipoic Acid | 15% |
| | polyvinyl pyrrolidone | 5% |
| | D calcium phosphate | 15% |
| | TOTAL | 100% |
| (14) | creatine ethyl ester | 33% |
| | Arginine α-ketoglutarate | 20% |
| | Lipoic Acid | 20% |
| | polyvinyl pyrrolidone | 5% |
| | D calcium phosphate | 12% |
| | TOTAL | 100% |
| (15) | creatine ethyl ester | 35% |
| | Arginine α-ketoglutarate | 20% |
| | Lipoic Acid | 20% |
| | polyvinyl pyrrolidone | 5% |
| | dibasic calcium phosphate | 15% |
| | Starch | 5% |
| | TOTAL | 100% |
| (16) | creatine ethyl ester | 35-45% |
| | Arginine α-ketoglutarate | 20-22.5% |
| | Lipoic Acid | 20-22.5% |
| | Hydroxyalkylcellulose | 10-20% |
| | Lactose | 5-10% |
| | microcrystalline cellulose | 4-6% |
| | titanium dioxide | 1-5% |
| | TOTAL | 100% |
| (17) | creatine ethyl ester | 40% |
| | Arginine α-ketoglutarate | 20% |
| | Lipoic Acid | 20% |
| | Alkylcellulose | 10% |
| | spray-dried lactose | 9.5% |
| | magnesium stearate | 0.5% |
| | TOTAL | 100% |
| (18) | creatine ethyl ester | 50% |
| | Arginine α-ketoglutarate | 15% |
| | Lipoic Acid | 15% |
| | carboxymethylcellulose (hydrogel matrix) | 10% |
| | polyethylene oxide (hydrogel matrix) | 10% |
| | TOTAL | 100% |
| (19) | creatine ethyl ester | 30% |
| | Arginine α-ketoglutarate | 30% |
| | Lipoic Acid | 20% |
| | polyvinylpyrrolidone (hydrogel matrix) | 5% |
| | polyethylene glycol (hydrogel matrix) | 15% |
| | TOTAL | 100% |
| (20) | creatine ethyl ester | 30-40% |
| | Arginine α-ketoglutarate | 20% |
| | Lipoic Acid | 20% |
| | hydroxypropyl methylcellulose | 5-10% |
| | Ethylcellulose | 5-10% |
| | Lactose | 5-15% |
| | Sorbitol | 4-6% |
| | silicon dioxide | 1-5% |
| | TOTAL | 100% |
| (21) | creatine ethyl ester | 35% |
| | Arginine α-ketoglutarate | 40% |
| | Lipoic Acid | 5% |
| | cellulose acetate butyrate | 10% |
| | Starch | 9.5% |
| | magnesium stearate | 0.5% |
| | TOTAL | 100% |
| (22) | creatine ethyl ester | 30% |
| | Arginine α-ketoglutarate | 10% |
| | Lipoic Acid | 30% |
| | cellulose acetate phthalate | 10% |
| | cellulose acetate trimellitate | 10% |
| | Mannitol | 9.5% |
| | calcium stearate | 0.5% |
| | TOTAL | 100% |
| (23) | creatine ethyl ester | 35% |
| | Arginine α-ketoglutarate | 25% |
| | Lipoic Acid | 20% |
| | polyvinylacetate phthalate | 5% |
| | hydroxypropylmethylcelluulose phthalate | 5% |
| | Sucrose | 5-9% |
| | stearic acid | 1-5% |
| | TOTAL | 100% |
| (24) | creatine ethyl ester | 35% |
| | Arginine α-ketoglutarate | 20% |
| | Lipoic Acid | 25% |
| | Methylcellulose | 10% |
| | hydroxypropylmethylcellulose | 5% |
| | Glucose | 4% |
| | Talc | 0.5% |
| | PEG 6000 | 0.5% |
| | TOTAL | 100% |
| (25) | Creatine ethyl ester | 30% |
| | Arginine α-ketoglutarate | 20% |
| | Lipoic Acid | 20% |
| | polyethylene glycol | 10% |
| | poly(alkyl methacrylate) | 10% |
| | calcium stearate | 5% |
| | dibasic calcium phosphate | 3% |
| | Poloxamers | 2% |
| | TOTAL | 100% |
| (26) | Creatine ethyl ester | 50% |
| | Arginine α-ketoglutarate | 15% |
| | Lipoic Acid | 15% |
| | Hydroxypropylmethylcellulose | 14% |
| | Pectin | 12% |
| | magnesium stearate | 4% |
| | TOTAL | 100% |
| (27) | Creatine ethyl ester | 36.7% |
| | Arginine α-ketoglutarate | 20% |
| | Lipoic Acid | 20% |
| | calcium sulfate | 7.3% |
| | Zein | 1.3% |
| | Alginate | 3.3% |
| | Pectin | 4.0% |
| | Glycerin | 6.7% |
| | magnesium stearate | 0.7% |
| | TOTAL | 100% |

In the above formulations any one of the active components could be replaced with a lipid soluble thiamine.

Oral dosage units comprising a creatine derivative are judged by many as having bitter favor. Thus, it is desirable to mask such which can be done by coating the dosage (e.g. tablet) with a dissolvable coating. Such a coating may be a pharmaceutical grade shellac or like material. The coating may add an additional 1% to 4% by weight to the dosage unit.

Those skilled in the art will recognize that there are endless possibilities in terms of formulations and that a margin of error e.g., ±20% or more preferably ±10%, should be accounted for with each component. Even if the formulations are limited to the relatively few compounds shown above, the formulation could be changed in limitless ways by adjusting the ratios of the components to each other.

A feature of an embodiment of a formulation of the invention may be designed so that the three active components be released in a controlled manner which makes it possible to maintain therapeutic levels of the active components over a substantially longer period of time as compared to a quick release formulation. A particularly preferred formulation will quickly obtain a therapeutic level and thereafter decrease the rate of release to closely match the rate at which the active components are metabolized thereby maintaining a therapeutic level in the patient over a maximum period of time based on the amount of active component in the oral dosage formulation. Some general types of controlled release technology which might be used with the present invention are described below followed by specific preferred formulations.

Formulations of the invention as described above are “quick release” formulations of active component and such provides a number of advantages. The creatine derivatives formulated in accordance with the present invention provide improved bioavailability as compared with creatine formulations. That improved bioavailability provides improved results in a number of areas as described here. However, formulations of the invention can be created so as to provide sustained release or controlled release of the active ingredient. When the active ingredient is maintained at therapeutic levels over longer periods of time results obtained are improved. Accordingly, the following provides information relating to the production of controlled release formulations.

### Four Component Formulations

A typical four component formulation of the invention will contain about 5% to about 50% by weight of each of the four active components and a particularly preferred formulation will comprise 10% to 30% ±10% by weight of each of the four active components. Assuming a formulation with about 25% ±10% by weight of each of the four active components with the remaining being the excipient material, there are a number of possible components and combinations thereof which could be used to make up the remainder of the formulation. The creatine derivative component is generally present in larger amounts and the lipid soluble thiamine component is generally present in smaller amounts relative to the other active components. A generalized and specific description of such is provided below:

| | | |
|---|---|---|
| (1) | Creatine ester | 30% |
| | Arginine α-ketoglutarate | 20% |
| | Lipoic Acid | 20% |
| | Lipid soluble thiamine | 10% |
| | biodegradable polymer | 20% |
| | TOTAL | 100% |
| (2) | Creatine ester | 30% |
| | Arginine α-ketoglutarate | 20% |
| | Lipoic Acid | 20% |
| | Lipid soluble thiamine | 10% |
| | biodegradable polymer | 14.5% |
| | Inorganics | 5.5% |
| | TOTAL | 100% |
| (3) | creatine ester | 25% |
| | Arginine α-ketoglutarate | 25% |
| | Lipoic Acid | 20% |
| | Lipid soluble thiamine | 10% |
| | organic polymer | 10%-20% |
| | Inorganics | 10% |
| | | or less |
| | TOTAL | 100% |
| (4) | creatine ester | 25% |
| | Arginine α-ketoglutarate | 25% |
| | Lipoic Acid | 20% |
| | Lipid soluble thiamine | 10% |
| | microcrystalline cellulose | 4% |
| | Cellulose acetate phthalate aqueous | 5% |
| | dispersion | |
| | Polyvinylpyrolidone | 3% |
| | ethyl acetate | 2.5% |
| | hydrous magnesium silicate (talc) | 1% |
| | carboxy methyl ether | 4% |
| | magnesium stearate | 0.5% |
| | TOTAL | 100% |
| (5) | creatine ester | 35% |
| | Arginine α-ketoglutarate | 20% |
| | Lipoic Acid | 15% |
| | Lipid soluble thiamine | 10% |
| | microcrystalline cellulose | 5-20% |
| | Cellulose acetate phthalate aqueous | 5-15% |
| | dispersion | |
| | polyvinylpyrolidone | 1-5% |
| | ethyl acetate | 1-5% |
| | hydrous magnesium silicate (talc) | 0.5-3% |
| | carboxy methyl ether | 1-5% |
| | magnesium stearate | 0.5-1.5% |
| | TOTAL | 100% |
| (6) | creatine ester | 30% |
| | Arginine α-ketoglutarate | 15% |
| | Lipoic Acid | 15% |
| | Lipid soluble thiamine | 10% |
| | microcrystalline cellulose, NF | 14% |
| | (Avicel PH 101) | |
| | Aquacoat CPD-30 (30% solids w/w) | 5% |
| | Plasdone K29/32, USP | 3% |
| | Carbopol 974P, NF | 2.5% |
| | Talc, USP | 1.0% |
| | croscarmellose sodium, NF (Ac, di-Sol) | 4.0% |
| | Magnesium Stearate, NF | 0.5% |
| | TOTAL | 100% |
| (7) | creatine ethyl ester | 25%-30% |
| | Arginine α-ketoglutarate | 20-22.5% |
| | Lipoic Acid | 20-22.5% |
| | Lipid soluble thiamine | 5-15% |
| | Diacalcium phosphate | 5-15% |
| | Polyvinyl pyrrolidone | 2-4% |
| | Starch | 2-4% |
| | Magnesium Stearate, NF | 0.5-1.5% |
| | TOTAL | 100% |
| (8) | creatine ethyl ester | 33% |
| | Arginine α-ketoglutarate | 20% |
| | Lipoic Acid | 25% |
| | Lipid soluble thiamine | 5% |
| | Di-Calcium Phosphate | 10% |
| | polyvinyl pyrrolidone (Kollidon 90) | 3% |
| | Starch | 3% |
| | Magnesium stearate | 1% |
| | TOTAL | 100% |
| (9) | creatine ethyl ester | 35% |
| | Arginine α-ketoglutarate | 15% |
| | Lipoic Acid | 15% |
| | Lipid soluble thiamine | 15% |
| | Poly-DL-lactide-co-glycolide (PLG) | 20% |
| | TOTAL | 100% |
| (10) | creatine ethyl ester | 25% |
| | Arginine α-ketoglutarate | 20% |
| | Lipoic Acid | 20% |
| | Lipid soluble thiamine | 5% |
| | hydroxypropyl methylcellulose | 20% |
| | Spray-dried lactose | 9.5% |
| | Magnesium stearate | 0.5% |
| | TOTAL | 100% |
| (11) | creatine ethyl ester | 30-35% |
| | Arginine α-ketoglutarate | 20-22.5% |
| | Lipoic Acid | 20-22.5% |
| | Lipid soluble thiamine | 5-15% |
| | Polyvinyl pyrrolidone (Kollidon 90) | 10-20% |
| | Lactose | 5-15% |
| | microcrystalline cellulose | 4-6% |
| | titanium dioxide | 1-5% |
| | TOTAL | 100% |
| (12) | creatine ethyl ester | 35% |
| | Arginine α-ketoglutarate | 15% |
| | Lipoic Acid | 15% |
| | Lipid soluble thiamine | 15% |
| | Polyvinyl pyrrolidone (Kollidon 90) | 20% |
| | TOTAL | 100% |
| (13) | creatine ethyl ester | 40% |
| | Arginine α-ketoglutarate | 20% |
| | Lipoic Acid | 15% |
| | Lipid soluble thiamine | 5% |
| | Polyvinyl pyrrolidone | 5% |
| | D calcium phosphate | 15% |
| | TOTAL | 100% |
| (14) | creatine ethyl ester | 33% |
| | Arginine α-ketoglutarate | 15% |
| | Lipoic Acid | 20% |
| | Lipid soluble thiamine | 5% |
| | Polyvinyl pyrrolidone | 5% |
| | D calcium phosphate | 12% |
| | TOTAL | 100% |
| (15) | creatine ethyl ester | 35% |
| | Arginine α-ketoglutarate | 15% |
| | Lipoic Acid | 15% |
| | Lipid soluble thiamine | 5% |
| | Polyvinyl pyrrolidone | 5% |
| | dibasic calcium phosphate | 15% |
| | Starch | 5% |
| | TOTAL | 100% |
| (16) | creatine ethyl ester | 35-45% |
| | Arginine α-ketoglutarate | 20-22.5% |
| | Lipoic Acid | 20-22.5% |
| | Lipid soluble thiamine | 5-15% |
| | hydroxyalkylcellulose | 10-20% |
| | Lactose | 5-10% |
| | microcrystalline cellulose | 4-6% |
| | titanium dioxide | 1-5% |
| | TOTAL | 100% |
| (17) | creatine ethyl ester | 30% |
| | Arginine α-ketoglutarate | 20% |
| | Lipoic Acid | 20% |
| | Lipid soluble thiamine | 10% |
| | Alkylcellulose | 10% |
| | spray-dried lactose | 9.5% |
| | magnesium stearate | 0.5% |
| | TOTAL | 100% |
| (18) | creatine ethyl ester | 30% |
| | Arginine α-ketoglutarate | 20% |
| | Lipoic Acid | 20% |
| | Lipid soluble thiamine | 10% |
| | carboxymethylcellulose (hydrogel matrix) | 10% |
| | polyethylene oxide (hydrogel matrix) | 10% |
| | TOTAL | 100% |
| (19) | creatine ethyl ester | 20% |
| | Arginine α-ketoglutarate | 30% |
| | Lipoic Acid | 20% |
| | Lipid soluble thiamine | 10% |
| | polyvinylpyrrolidone (hydrogel matrix) | 5% |
| | polyethylene glycol (hydrogel matrix) | 15% |
| | TOTAL | 100% |
| (20) | creatine ethyl ester | 15-45% |
| | Arginine α-ketoglutarate | 15% |
| | Lipoic Acid | 15% |
| | Lipid soluble thiamine | 10% |
| | hydroxypropyl methylcellulose | 5-10% |
| | Ethylcellulose | 5-10% |
| | Lactose | 5-15% |
| | Sorbitol | 4-6% |
| | silicon dioxide | 1-5% |
| | TOTAL | 100% |
| (21) | creatine ethyl ester | 20% |
| | Arginine α-ketoglutarate | 40% |
| | Lipoic Acid | 5% |
| | Lipid soluble thiamine | 15% |
| | cellulose acetate butyrate | 10% |
| | Starch | 9.5% |
| | magnesium stearate | 0.5% |
| | TOTAL | 100% |
| (22) | creatine ethyl ester | 30% |
| | Arginine α-ketoglutarate | 10% |
| | Lipoic Acid | 20% |
| | Lipid soluble thiamine | 10% |
| | cellulose acetate phthalate | 10% |
| | cellulose acetate trimellitate | 10% |
| | Mannitol | 9.5% |
| | calcium stearate | 0.5% |
| | TOTAL | 100% |
| (23) | creatine ethyl ester | 35% |
| | Arginine α-ketoglutarate | 15% |
| | Lipoic Acid | 20% |
| | Lipid soluble thiamine | 10% |
| | polyvinylacetate phthalate | 5% |
| | hydroxypropylmethylcelluulose phthalate | 5% |
| | Sucrose | 5-9% |
| | stearic acid | 1-5% |
| | TOTAL | 100% |
| (24) | creatine ethyl ester | 35% |
| | Arginine α-ketoglutarate | 20% |
| | Lipoic Acid | 20% |
| | Lipid soluble thiamine | 5% |
| | Methylcellulose | 10% |
| | hydroxypropylmethylcellulose | 5% |
| | Glucose | 4% |
| | Talc | 0.5% |
| | PEG 6000 | 0.5% |
| | TOTAL | 100% |
| (25) | Creatine ethyl ester | 20% |
| | Arginine α-ketoglutarate | 20% |
| | Lipoic Acid | 20% |
| | Lipid soluble thiamine | 10% |
| | polyethylene glycol | 10% |
| | poly(alkyl methacrylate) | 10% |
| | calcium stearate | 5% |
| | dibasic calcium phosphate | 3% |
| | Poloxamers | 2% |
| | TOTAL | 100% |
| (26) | Creatine ethyl ester | 35% |
| | Arginine α-ketoglutarate | 15% |
| | Lipoic Acid | 15% |
| | Lipid soluble thiamine | 15% |
| | Hydroxypropylmethylcellulose | 14% |
| | Pectin | 12% |
| | magnesium stearate | 4% |
| | TOTAL | 100% |
| (27) | Creatine ethyl ester | 36.7% |
| | Arginine α-ketoglutarate | 20% |
| | Lipoic Acid | 10% |
| | Lipid soluble thiamine | 10% |
| | calcium sulfate | 7.3% |
| | Zein | 1.3% |
| | Alginate | 3.3% |
| | Pectin | 4.0% |
| | Glycerin | 6.7% |
| | magnesium stearate | 0.7% |
| | TOTAL | 100% |

Oral dosage units comprising a creatine derivative are judged by many as having bitter favor. Thus, it is desirable to mask such which can be done by coating the dosage (e.g. tablet) with a dissolvable coating. Such a coating may be a pharmaceutical grade shellac or like material. The coating may add an additional 1% to 4% by weight to the dosage unit.

Those skilled in the art will recognize that there are endless possibilities in terms of formulations and that a margin of error e.g., ±20% or more preferably ±10% (by weight, based on the weight of the component), should be accounted for with each component. Even if the formulations are limited to the relatively few compounds shown above, the formulation could be changed in limitless ways by adjusting the ratios of the components to each other.

A feature of an embodiment of a formulation of the invention is that the active components be released in a controlled manner which makes it possible to maintain therapeutic levels of the active components over a substantially longer period of time as compared to a quick release formulation. A particularly preferred formulation will quickly obtain a therapeutic level and thereafter decrease the rate of release to closely match the rate at which the active components are metabolized thereby maintaining a therapeutic level in the patient over a maximum period of time based on the amount of active component in the oral dosage formulation. Some general types of controlled release technology which might be used with the present invention are described below followed by specific preferred formulations.

Formulations of the invention as described above are “quick release” formulations of active component and such provides a number of advantages. The creatine derivatives formulated in accordance with the present invention provide improved bioavailability as compared with creatine formulations. That improved bioavailability provides improved results in a number of areas as described here. However, formulations of the invention can be created so as to provide sustained release or controlled release of the active ingredient. When the active ingredient is maintained at therapeutic levels over longer periods of time results obtained are improved. Accordingly, the following provides information relating to the production of controlled release formulations.

Those skilled in the art will recognize that there are endless possibilities in terms of formulations and that a margin of error e.g. ±20% or ±10% or ±5% (by weight) should be accounted for with each component. Even if the formulations are limited to the relatively few compounds shown above the formulation could be changed in limitless ways by adjusting the ratios of the components to each other. An important feature of any formulation of the invention is that the active components each be present in a therapeutically effective amount. It may be important that the lipoic acid be released in a controlled manner which makes it possible to maintain therapeutic levels of lipoic acid over a substantially longer period of time as compared to a quick release formulation. A particularly preferred formulation will quickly obtain a therapeutic level of all of the active components and thereafter decrease the rate of release to closely match the rate at which each of the active components are being metabolized thereby maintaining a therapeutic level in the patient over a maximum period of time based on the amount of active components in the oral dosage formulation. Some general types of controlled release technology which might be used with the present invention are described below followed by specific preferred formulations.

### Controlled Release Technology

Controlled release within the scope of this invention can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

There are corporations with specific expertise in drug delivery technologies including controlled release oral formulations such as Alza corporation and Elan. A search of patents, published patent applications and related publications will provide those skilled in the art reading this disclosure with significant possible controlled release oral formulations. Examples include the formulations disclosed in any of the U.S. Pat. No. 5,637,320 issued Jun. 10, 1997; U.S. Pat. No. 5,505,962 issued Apr. 9, 1996; U.S. Pat. No. 5,641,745 issued Jun. 24, 1997; U.S. Pat. No. 5,641,515 issued Jun. 24, 1997; U.S. Pat. No. 6,572,888 issued Jun. 3, 2003; and U.S. Pat. No. 6,191,162 issued Feb. 20, 2001. Although specific formulations are disclosed here and in these patents the invention is more general than any specific formulation. This includes the discovery that by placing lipoic acid in a controlled release formulation which maintains therapeutic levels over substantially longer periods of time as compared to quick release formulations, improved unexpected results are obtained.

The various controlled release technologies cover a very broad spectrum of drug dosage forms. Controlled release technologies include, but are not limited to physical systems and chemical systems.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.).

Controlled release drug delivery systems may also be categorized under their basic technology areas, including, but not limited to, rate-preprogrammed drug delivery systems, activation-modulated drug delivery systems, feedback-regulated drug delivery systems, and site-targeting drug delivery systems.

In rate-preprogrammed drug delivery systems, release of drug molecules from the delivery systems “preprogrammed” at specific rate profiles. This may be accomplished by system design, which controls the molecular diffusion of drug molecules in and/or across the barrier medium within or surrounding the delivery system. Fick's laws of diffusion are often followed.

In activation-modulated drug delivery systems, release of drug molecules from the delivery systems is activated by some physical, chemical or biochemical processes and/or facilitated by the energy supplied externally. The rate of drug release is then controlled by regulating the process applied, or energy input.

In feedback-regulated drug delivery systems, release of drug molecules from the delivery systems may be activated by a triggering event, such as a biochemical substance, in the body. The rate of drug release is then controlled by the concentration of triggering agent detected by a sensor in the feedback regulated mechanism.

In a site-targeting controlled-release drug delivery system, the drug delivery system targets the active molecule to a specific site or target tissue or cell. This may be accomplished, for example, by a conjugate including a site specific targeting moiety that leads the drug delivery system to the vicinity of a target tissue (or cell), a solubilizer that enables the drug delivery system to be transported to and preferentially taken up by a target tissue, and a drug moiety that is covalently bonded to the polymer backbone through a spacer and contains a cleavable group that can be cleaved only by a specific enzyme at the target tissue.

While a preferable mode of controlled release drug delivery will be oral, other modes of delivery of controlled release compositions according to this invention may be used. These include mucosal delivery, nasal delivery, ocular delivery, transdermal delivery, parenteral controlled release delivery, vaginal delivery, and intrauterine delivery.

There are a number of controlled release drug formulations that are developed preferably for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs. Additional information regarding controlled release drug delivery systems may be found in Yie W. Chien, Novel Drug Delivery Systems, 1992 (Marcel Dekker, Inc.). some of these formulations will now be discussed in more detail.

Enteric coatings are applied to tablets to prevent the release of drugs in the stomach either to reduce the risk of unpleasant side effects or to maintain the stability of the drug which might otherwise be subject to degradation of expose to the gastric environment. Most polymers that are used for this purpose are polyacids that function by virtue or the fact that their solubility in aqueous medium is pH-dependent, and they require conditions with a pH higher then normally encountered in the stomach.

One preferable type of oral controlled release structure is enteric coating of a solid or liquid dosage form. Enteric coatings promote the lipoates' remaining physically incorporated in the dosage form for a specified period when exposed to gastric juice. Yet the enteric coatings are designed to disintegrate in intestinal fluid for ready absorption. Delay of the lipoates' absorption is dependent on the rate of transfer through the gastrointestinal tract, and so the rate of gastric emptying is an important factor. Some investigators have reported that a multiple-unit type dosage form, such as granules, may be superior to a single-unit type. Therefore, in a preferable embodiment, the lipoates may be contained in an enterically coated multiple-unit dosage form. In a more preferable embodiment, the lipoate dosage form is prepared by spray-coating granules of an lipoate-enteric coating agent solid dispersion on an inert core material. These granules can result in prolonged absorption of the drug with good bioavailability.

Typical enteric coating agents include, but are not limited to, hydroxypropylmethylcellulose phthalate, methacryclic acid-methacrylic acid ester copolymer, polyvinyl acetate-phthalate and cellulose acetate phthalate. Akihiko Hasegawa, Application of solid dispersions of Nifedipine with enteric coating agent to prepare a sustained-release dosage form, Chem. Pharm. Bull. 33: 1615-1619 (1985). Various enteric coating materials may be selected on the basis of testing to achieve an enteric coated dosage form designed ab initio to have a preferable combination of dissolution time, coating thicknesses and diametral crushing strength. S. C. Porter et al., The Properties of Enteric Tablet Coatings Made From Polyvinyl Acetate-phthalate and Cellulose acetate Phthalate, J. Pharm. Pharmacol. 22:42p (1970).

On occasion, the performance of an enteric coating may hinge on its permeability. S. C. Porter et al., The Permeability of Enteric Coatings and the Dissolution Rates of Coated Tablets, J. Pharm. Pharmacol. 34: 5-8 (1981). With such oral drug delivery systems, the drug release process may be initiated by diffusion of aqueous fluids across the enteric coating. Investigations have suggested osmotic driven/rupturing affects as important release mechanisms from enteric coated dosage forms. Roland Bodmeier et al., Mechanical Properties of Dry and Wet Cellulosic and Acrylic Films Prepared from Aqueous Colloidal Polymer Dispersions used in the Coating of Solid Dosage Forms, Pharmaceutical Research, 11: 882-888 (1994).

Another type of useful oral controlled release structure is a solid dispersion. A solid dispersion may be defined as a dispersion of one or more active ingredients in an inert carrier or matrix in the solid state prepared by the melting (fusion), solvent, or melting-solvent method. Akihiko Hasegawa, Super Saturation Mechanism of Drugs from Solid Dispersions with Enteric Coating Agents, Chem. Pharm. Bull. 36: 4941-4950 (1998). The solid dispersions may be also called solid-state dispersions. The term “coprecipitates” may also be used to refer to those preparations obtained by the solvent methods.

Solid dispersions may be used to improve the solubilities and/or dissolution rates of poorly water-soluble lipoates. Hiroshi Yuasa, et al., Application of the Solid Dispersion Method to the Controlled Release Medicine. III. Control of the Release Rate of Slightly Water-Soluble Medicine From Solid Dispersion Granules, Chem. Pharm. Bull. 41:397-399 (1993). The solid dispersion method was originally used to enhance the dissolution rate of slightly water-soluble medicines by dispersing the medicines into water-soluble carriers such as polyethylene glycol or polyvinylpyraolidone, Hiroshi Yuasa, et al., Application of the Solid Dispersion Method to the Controlled Release of Medicine. IV. Precise Control of the Release Rate of a Water-Soluble Medicine by Using the Solid Dispersion Method Applying the Difference in the Molecular Weight of a Polymer, Chem. Pharm. Bull. 41:933-936 (1993).

The selection of the carrier may have an influence on the dissolution characteristics of the dispersed drug because the dissolution rate of a component from a surface may be affected by other components in a multiple component mixture. For example, a water-soluble carrier may result in a fast release of the drug from the matrix, or a poorly soluble or insoluble carrier may lead to a slower release of the drug from the matrix. The solubility of the lipoates may also be increased owing to some interaction with the carriers.

Examples of carriers useful in solid dispersions according to the invention include, but are not limited to, water-soluble polymers such as polyethylene glycol, polyvinylpyraolidone, or hydroxypropylmethyl-cellulose. Akihiko Hasegawa, Application of Solid Dispersions of Nifedipine with Enteric Coating Agent to Prepare a Sustained-release Dosage Form, Chem. Pharm. Bull. 33: 1615-1619 (1985).

Alternate carriers include phosphatidylcholine. Makiko Fujii, et al., The Properties of Solid Dispersions of Indomethacin, Ketoprofen and Flurbiprofen in Phosphatidylcholine, Chem. Pharm. Bull. 36:2186-2192 (1988). Phosphatidylcholine is an amphoteric but water-insoluble lipid, which may improve the solubility of otherwise insoluble lipoates in an amorphous state in phosphatidylcholine solid dispersions. See Makiko Fujii, et al., Dissolution of Bioavailibility of Phenytoin in Solid Dispersion with Phosphatidylcholine, Chem. Pharm. Bull. 36:4908-4913 (1988).

Other carriers include polyoxyethylene hydrogenated castor oil. Katsuhiko Yano, et al., In-Vitro Stability and In-Vivo Absorption Studies of Colloidal Particles Formed From a Solid Dispersion System, Chem. Pharm. Bull 44:2309-2313 (1996). Poorly water-soluble lipoates may be included in a solid dispersion system with an enteric polymer such as hydroxypropylmethylcellulose phthalate and carboxymethylethylcellulose, and a non-enteric polymer, hydroxypropylmethylcellulose. See Toshiya Kai, et al., Oral Absorption Improvement of Poorly Soluble Drug Using Soluble Dispersion Technique, Chem. Pharm. Bull. 44:568-571 (1996). Another solid dispersion dosage form include incorporation of the drug of interest with ethyl cellulose and stearic acid in different ratios. Kousuke Nakano, et al., Oral Sustained-Release Cisplatin Preparations for Rats and Mice, J. Pharm. Pharmacol. 49:485-490 (1997).

The active component of the invention can be incorporated into any one of the aforementioned controlled released dosage forms, or other conventional dosage forms. The amount of each active component contained in each dose can be adjusted, to meet the needs of the individual patient, and the indication. One of skill in the art and reading this disclosure will readily recognize how to adjust the level of each active component and the release rates in a controlled release formulation, in order to optimize delivery of each active component and its bioavailability.

### Therapeutic Indications/Lipoic Acid

Formulations of the present invention can be used to obtain a wide range of desirable effects. Particularly the formulations of the invention are useful in treating essentially any disease state or symptom which is treatable by long term administration of antioxidants. Further, formulations of the invention can be used in treating patients with abnormally low levels of thiamine or vitamin B1. Still further, the invention can be used in the treatment of diseases which involve carbohydrate metabolism and blood glucose disposal which includes various forms of diabetes. In addition, the inventions can be used in the treatment of diabetic polyneuropathy. Further, the invention is useful in the treatment of various adverse effects on the eyes and skin when the adverse effect are due to high levels of free radicals which can be dissipated by the presence of antioxidants or high levels of serum glucose which can be reduced by stimulating basal glucose transport. Maintaining substantially constant levels of lipoic acid provides a long term antioxidant effect which assists in immunomodulation and can result in improved liver and kidney function. Because of the long term antioxidant effect in the circulatory system the present invention has a variety of beneficial effects on the cardiovascular system. Administering the lipid soluble thiamine is useful in the alleviation of certain liver diseases as well as neurodegenerative diseases related to diabetes. A patient infected with HIV can benefit from the enhanced effect obtained on the immune system.

Because of the very minimal toxicity of the active components the formulation can be given to a wide range of patients which have different conditions from mild to serious without fear of adverse effects. Further, the controlled release formulations taught here are even safer than quick release formulations in that serum levels obtained are low compared to quick release formulations. One mild side effect experienced by some patients taking controlled release lipoic acid is mild headaches over the first few days. The headaches have not been observed with quick release formulations of lipoic acid. Patients treated with vasodilators experience the same mild headaches over the first days of treatment. The headaches are believed to be caused by the vasodilator effect allowing increased blood flow to the brain. Accordingly, controlled release formulations of the invention can be used as a vasodilator to treat patients with angina.

The data provided here do not show specific treatments of many of the diseases or symptoms mentioned above. However, the invention is believed to be responsible for obtaining a wide range of beneficial effects particularly when the controlled release formulation is administered to patient's (e.g. on consecutive days) over long periods of time, i.e. weeks, months and years. By maintaining substantially constant therapeutic levels of the active components in the blood over very long periods of time a range of desirable physiological results are obtained. Stated differently by continually maintaining the constant therapeutic serum levels of an active component such as lipoic acid and keeping a patient's blood glucose level within a more desirable range the adverse effects obtained from free radicals and high fluctuating glucose levels are avoided.

### Therapeutic Indications/Thiamine

There is no known toxicity in humans from thiamine taken orally. People have taken hundreds of milligrams daily without any harmful effect, although some may become more stimulated than others. Thiamine injections, however, have occasionally been associated with trauma or edema.

Prolonged restriction of thiamine intake may produce a wide variety of symptoms, particularly affecting the general disposition, nervous system, gastrointestinal tract, and heart. With thiamine deficiency, as with deficiency of most any essential nutrient, symptoms range from mild to moderate depletion disorders to the serious disease state that RDA amounts usually prevent.

Beriberi is the name given to the disease caused by thiamine deficiency. There are three basic expressions of beriberi, namely childhood, wet, and dry beriberi. Childhood beriberi stunts the growth process, and in infants high-pitched scream and rapid heartbeat are associated with the disease. Wet beriberi is the classic form with edema (swelling) in the feet and legs, spreading to the body, and associated decreased function of the heart. Dry beriberi is not accompanied by swelling but seems to be manifested by weight loss, muscle wasting, and nerve degeneration. Another thiamine deficiency disease involves degeneration of the brain and affects the general orientation, attitude, and ability to walk. This has been termed the Wernicke-Korsakoff syndrome and is usually seen in people who have been addicted to alcohol for many years.

These severe problems can and do lead to death when they are not corrected with dietary change or supplemental thiamine. Before vitamin B1 was discovered, this affected many people who ate a diet consisting mainly of polished rice. Today, deficiency of this vitamin is still quite common. Although it does not usually lead to beriberi, a number of symptoms can result from a depletion of thiamine body levels. A low-B1 diet consisting of polished rice or unenriched white flour is not often the culprit in our culture. The diet that contributes to deficiency today, especially among teenagers, is high in colas, sweets, fast foods, and many other empty-calorie foods. This diet can also lead to skin problems and symptoms of neurosis, almost like a Jekyll-and-Hyde disposition.

With a deficiency of thiamine, carbohydrate digestion and the metabolism of glucose are diminished. There is a build-up of pyruvic acid in the blood, which can lead to decreased oxygen utilization and therefore mental deficiency and even difficulty in breathing. While B1 is needed for alcohol metabolism, alcohol abuse is often associated with a poor diet and poor B1 absorption. The poor perceptions, mental states, and nerve problems that come with alcoholism may be associated with thiamine deficiency.

The first symptoms of thiamine deficiency may be fatigue, instability. These may be followed by confusion, loss of memory, depression, clumsiness, insomnia, gastrointestinal disturbances, abdominal pain, constipation, slow heart rate, and burning chest pains. As the condition progresses, there may be problems of irregular heart rhythm, prickling sensation in the legs, loss of vibratory sensation, and the muscles may become tender and atrophy. The optic nerve may become inflamed and the vision will be affected.

Generally, with low B1 the central nervous system—the brain and nerves—does not function optimally. The gastrointestinal and cardiovascular systems are also influenced greatly. Vitamin B1 levels have been shown to be low in many elderly people, especially those that experience senility, neuroses, and schizophrenia.

Since thiamine is eliminated through the skin somewhat, doses of over 50-100 mg. per day may help repel insects such as flies and mosquitos from those with “sweet blood.” Other uses for increased thiamine include treatment of stress and muscle tensions, diarrhea, fever and infections, cramps, and headaches.

Thiamine needs are also increased with higher stress levels, with fever or diarrhea, and during and after surgery. Those who smoke, drink alcohol, consume caffeine or tannin from coffee or tea, or who are pregnant, lactating, or taking birth control pills all need more thiamine, possibly much more than the RDA, for optimum health.

Thiamine is needed in the diet or in supplements daily. There are some stores in the heart, liver, and kidneys; however, these do not last very long. The minimum B1 intake for those who are very healthy is at least 2 mg. per day. A good insurance level of thiamine is probably 10 mg. a day, though even higher levels may be useful in some situations. When we do not eat optimally, have any abusive substance habits (especially alcohol abuse), or are under stress, increased levels of thiamine are recommended. An example is the B complex 50 products—that is, 50 mg. of B1 along with that amount of most of the other B vitamins—suggested as a daily regimen. The upper intake levels of thiamine should not be much more than 200-300 mg. daily. Often B1, B2 (riboflavin), and B6 (pyridoxine) are formulated together in equal amounts within a B-complex supplement. When people take higher amounts of the B vitamins, many feel a difference in energy and vitality. (Note: Riboflavin taken for any length of time is best limited to 50 mg. daily.)

### Therapeutic Indications/Arginine

The arginine α-ketoglutarate formulations of the present invention can be used to obtain a wide range of desirable effects. Further, the invention can be used in the treatment of diseases which involve glutamate dehydrogenase deficiency, depressed prolyl hydroxylase and lysyl hydroxylase activity. Further, the invention is useful in the treatment of various adverse effects on the eyes and skin when the adverse effect are due to accumulation of protein glycation. Maintaining substantially constant levels of arginine α-ketoglutarate provides a long term antioxidant effect which assists in immunomodulation.

Because of the very minimal toxicity of arginine α-ketoglutarate, it can be given to a wide range of patients which have different conditions from mild to serious without fear of adverse effects. Further, the controlled release formulations taught here are even safer than quick release formulations in that serum levels obtained are low compared to quick release formulations.

However, the invention is believed to be responsible for obtaining a wide range of beneficial effects particularly when the formulation is administered to patients over long periods of time, i.e., weeks, months and years. By maintaining substantially constant therapeutic levels of arginine α-ketoglutarate in the blood over very long periods of time a range of desirable physiological results are obtained. Stated differently, by continually maintaining the constant therapeutic serum levels of the powerful antioxidant and preventing protein glycation, the pathogenesis of atherosclerosis, cataracts and retinopathy is prevented. Overall circulation can be improved and antioxidant effects obtained.

### Therapeutic Indications/Creatine Derivatives

The creatine ester formulations of the present invention can be used to obtain a wide range of desirable effects. Formulations of the invention may be administered to patients having myoclonus (i.e., a neuromuscular disorder characterized by the occurrence of irregular, asynergic, and jactitious contractions of muscles producing non repetitive, brief, involuntary movements in various body areas) as a symptom of epilepsy, neurodegenerative disease such as Parkinson's disease, multiple sclerosis or amyotrophic lateral sclerosis (ALS) and Tourette's syndrome. Further, the invention can be used to enhance muscle performance.

Because of the very minimal toxicity of creatine ester, it can be given to a wide range of patients which have different conditions from mild to serious without fear of adverse effects. Further, the controlled release formulations taught here are even safer than quick release formulations in that serum levels obtained are low compared to quick release formulations.

The data provided here do not show specific treatments of many of the diseases or symptoms mentioned above. However, the invention is believed to be responsible for obtaining a wide range of beneficial effects particularly when the controlled release formulation is administered to patients over long periods of time on a daily basis for weeks, months and years. By maintaining substantially constant therapeutic levels of creatine in the blood over very long periods of time a range of desirable physiological results are obtained. For example, by continually maintaining the constant therapeutic serum levels of creatine muscle performance is enhanced.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An oral dosage formulation, comprising:
an excipient material and any two or more of active components chosen from:
a therapeutically effective amount of arginine α-ketoglutarate;
a therapeutically effective amount of a creatine derivative;
a therapeutically effective amount of lipoic acid; and
a therapeutically effective amount of a lipid soluble thiamine.

2. The formulation of claim 1, wherein the lipoic acid comprises a racemic mixture of enantiomers.

3. The formulation of claim 1, wherein the creatine derivative is a creatine ester.

4. The formulation of claim 3, wherein the creatine ester is creatine ethyl ester.

5. The formulation of claim 1, wherein the formulation is characterized by releasing a first portion of the lipoic acid sufficient to obtain a therapeutic level at a first rate substantially equivalent to a release rate of a quick release formulation and releasing a remaining portion of the lipoic acid at a controlled rate which is below a release rate of a quick release formulation.

6. The formulation of claim 5, wherein the first portion of the lipoic acid is from about 10% to about 50% of the lipoic acid in the formulation.

7. The formulation of claim 1, wherein the formulation comprises any three of the active components.

8. The formulation of claim 1, wherein the formulation comprises all four of the active components.

9. The formulation of claim 1, wherein the lipid soluble thiamine is chosen from benfotiamine and prosultamine.

10. The formulation of claim 8, further comprising an orally active antidiabetic chosen from a sulfonylurea, a biguanide and a thiazolidinedione.

11. The formulation of claim 8, further comprising metformin hydrochloride.

12. The formulation of claim 1, wherein the lipoic acid is present as a racemic mixture of R-(+) and S-(−) enantiomers and the therapeutic level is maintained over a period of four hours or more.

13. The formulation of claim 1, wherein the lipoic acid is present as substantially pure R-(+) enantiomer and the therapeutic level is maintained over a period of four hours or more and further wherein the lipid soluble thiamine is chosen from benfotiamine and prosultamine.

14. A method of treatment, comprising:
orally administering to a patient a formulation comprising two or more active components chosen from a lipid soluble thiamine, lipoic acid, arginine α-ketoglutarate and a creatine derivative; and
repeating the administering on three or more consecutive days thereby maintain a therapeutic level of active components in the patient's circulatory system over a therapeutically effective period of time on three or more consecutive days.

15. The method of claim 14, wherein the therapeutic level is maintained over a period of time which is 10% or more than that obtained with a quick release formulation and further wherein the repeating is over thirty or more consecutive days.

16. The method of claim 14, wherein the therapeutic level is maintained over a period of time which is 100% or more than that obtained with a quick release formulation and further wherein the repeating is over thirty or more consecutive days.

17. The method of claim 16, wherein one of the active components is lipoic acid and the therapeutic level of lipoic acid is a level sufficient to obtain measurable vasodilation in a human patient.

18. The method of claim 17, wherein the therapeutic level is a level sufficient to obtain a measurable reduction in a human patient's serum glucose level of 10% or more compared to a level prior to administering the formulation.

19. The method of claim 14, further comprising:
repeatedly administering the formulation on a daily basis for five or more days.

20. A method of treating a human patient, comprising:
administering to a human patient a formulation comprising active components of a lipid soluble thiamine, lipoic acid, arginine α-ketoglutarate, and a creatine derivative which formulation is characterized by maintaining a therapeutic level of the active components in the patient's circulatory system over a therapeutically effective period of time; and
repeating the administering on three or more consecutive days thereby maintaining a therapeutic level of the active components in the patient's circulatory system over a therapeutically effective period of time on three or more consecutive days.
